(19) United States
(12) Patent Application Publication
(10) Pub. No.: US 2025/0320525 A1
(43) Pub. Date: Oct. 16, 2025

(54) POLYMERIC SYSTEM FOR GENE DELIVERY

(71) Applicant: REGENTS OF THE UNIVERSITY OF MINNESOTA, Minneapolis, MN (US)

(72) Inventors: Theresa REINEKE, Vadnais Heights, MN (US); Rishad DALAL, Minneapolis, MN (US)

(21) Appl. No.: 18/865,404

(22) PCT Filed: May 15, 2023
(86) PCT No.: PCT/US2023/022253

§ 371 Date: Nov. 13, 2024

Related U.S. Application Data
(60) Provisional application No. 63/341,768, filed on May 13, 2022.

(51) Int. Cl. C12N 15/87; C08F 8/32; C08F 8/34; C12N 9/22; C12N 15/11; G16C 20/70

(52) U.S. Cl. CPC C12N 15/87; C08F 8/32; C08F 8/34; C12N 9/226; C12N 15/11; G16C 20/70; C12N 2310/20

(57) ABSTRACT

This invention relates generally to polymeric delivery systems. The present polymeric delivery systems may be complexed with biological agents, including nucleic acids, peptides, proteins, or small molecules, for delivery to cells. In particular, the present polymeric delivery systems may be used in gene editing.

### BACKGROUND OF THE INVENTION

Delivery of biological agents, such as nucleic acids, peptides, proteins, or small molecules, to cells both in vitro and in vivo has been performed using various recombinant viral vectors, lipid delivery systems, and electroporation. Such techniques have sought to treat various diseases and disorders by reducing or inhibiting gene expression, providing genetic constructs for gene therapy or to study various biological systems.

Genome editing, for example, based on clustered regularly interspersed palindromic repeats (CRISPR) technology has transformed the therapeutic landscape for diseases wherein the deletion, insertion or repair of genetic sequences can restore healthy cellular states. Clinical trials of investigational gene therapeutics for β-thalassemia and sickle cell disease suggest that safe and efficacious treatment is possible using CRISPR-based genome editing technology. Additional clinical trials are underway to develop CRISPR-based therapeutics for debilitating conditions such as Duchenne's muscular dystrophy (DMD), Leber congenital amaurosis (LCA) and for chimeric antigen receptor T-cell (CAR-T) therapies for cancer.

Despite the vast curative potential of CRISPR, widespread clinical deployment faces an uncertain outlook due to reliance on engineered viral vectors, which can be used to deliver therapeutic biomacromolecule payloads such as messenger RNA (mRNA), plasmid DNA (pDNA) and small interfering RNA (siRNA). However, the high costs, lengthy time requirements, and regulatory challenges involved in manufacturing clinical grade viruses at scale for large patient populations have imposed severe logistical bottlenecks. In addition to manufacturing and regulatory delays, the cargo capacity of viral vectors is limited, and this size restriction is particularly problematic in the context of bulky multi-component CRISPR cargoes.

Although advances in virus manufacturing have minimized occurrences of carcinogenic mutations, genomic integration and fatal systemic inflammatory responses, these risks are amplified when repeated dosing or large dosages are involved. For CRISPR therapeutics to become safe, scalable, and affordable, there is a need to identify synthetic substitutes for viral carriers.

Polymeric delivery vehicles have been used in clinical therapies due to their versatility, relative low production cost, and low immunogenicity. Synthetic polymers have been used to deliver biomacromolecule payloads such as, for example, pDNA, ribonucleoproteins (RNP), and the like, due to their versatility, low toxicity, and the ability to encapsulate large payloads. Some recent examples indicate that synthetic polymer-based systems achieved biomacromolecule based gene delivery and gene editing both in vitro and in vivo.

For example, in aqueous physiological solutions, cationic polymers can spontaneously bind with negatively charged pDNA and form interpolyelectrolyte complexes. These complexes are predominately internalized by various endocytic routes, followed by cargo release from these vesicles inside the cells via different proposed mechanisms, and subsequent entry into the cell nucleus to promote gene expression. Compared to viral vehicles, polymeric delivery systems typically have lower delivery efficiency, and various optimization strategies can be used to improve this parameter such as changing the cationic moieties on polymers, adding targeting ligands, and installing responsive monomers, which can improve uptake efficiency and help to balance transfection efficiency and cytotoxicity. However, their utility in genome editing is relatively underexplored.

Novel and efficient polymer-based delivery vehicles are thus desired.

### SUMMARY OF THE INVENTION

The present invention is related to polymeric delivery systems. The present polymeric delivery systems may be complexed with biological agents, including nucleic acids, peptides, proteins, or small molecules, for delivery to cells.

In one aspect, the invention features a polymer of formula (I)

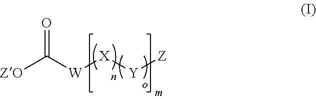

wherein W is alkylene; each X is independently

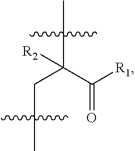

wherein R₁ is —O—C₆F₅ or —NR′R″,
wherein R′ and R″ are each independently H, alkenyl, or substituted alkylthio alkyl, and wherein R₂ is H or CH₃; each Y is independently

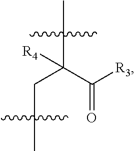

wherein R₃ is —NR′R″, wherein R′ and R″ are each independently H, alkenyl, or substituted alkylthio alkyl, and wherein R₄ is H or CH₃; Z is hydrogen, alkyl, thiol, or

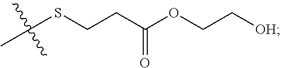 [SMILES: *SCCC(=O)OCCO]

Z′ is H or alkyl; m is from 1 to 1000; each of n and o represents a fraction of m, and the sum of n and o is 1; or an ion or salt thereof.

In some embodiments, at least one X or Y

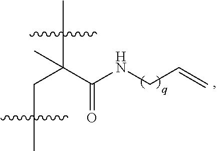

wherein q is 1 to 4.

In some embodiments, at least one X or Y is

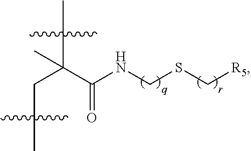

wherein R₅ is NRₐR_{b}, where Rₐ and R_{b} are independently H or alkyl, and q and r are independently 1 to 6. In some embodiments, at least one X or Y is

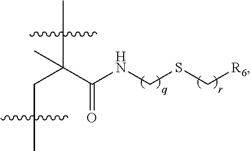

wherein R₆ is a basic nitrogen containing heterocycle, and q and r are independently 1 to 6.

In some embodiments, the basic nitrogen containing heterocycle is

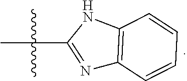 [SMILES: *c1nc2ccccc2[nH]1]

In some embodiments, W is substituted with cyano.

In another aspect, the invention features a compound of formula (II)

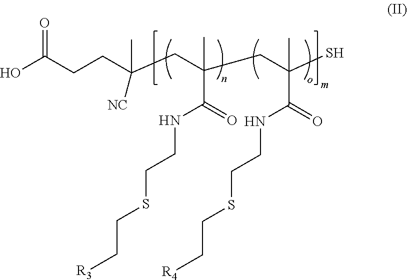

wherein R₃ is NH₃, N(CH₃)₂, or N(C₂H₅)₂; R₄ is a basic nitrogen containing heterocycle; m is from 1 to 1000; each of n and o represents a fraction of m, and the sum of n and o is 1; or a salt or ion thereof.

In some embodiments, the basic nitrogen containing heterocycle is

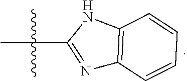 [SMILES: *c1nc2ccccc2[nH]1]

In some embodiments, m is from 1 to 500. In some embodiments, m is from 50 to 300. In some embodiments, n is from 0.5 to 1. In some embodiments, n is from 0.7 to 1.

In another aspect, the invention features a complex including a first polymer of any one of the presently described polymers and a negatively charged biological agent. In certain embodiments, the complex further includes a second polymer of any one of the presently described polymers.

In some embodiments, the negatively charged biological agent includes a nucleic acid. In some embodiments, the nucleic acid includes DNA or RNA. In some embodiments, the nucleic acid includes gRNA, mRNA, tmRNA, tRNA, rRNA, siRNA, shRNA, PNA, ssRNA, dsRNA, pDNA, ssDNA, dsDNA, a DNA:RNA hybrid molecule, a plasmid, an artificial chromosome, cDNA, a PCR product, a restriction fragment, a ribozyme, an antisense construct, or a combination thereof.

In some embodiments, the negatively charged biological agent includes a protein. In some embodiments, the protein includes a ribonucleoprotein. In some embodiments, the ribonucleoprotein includes a virus, a ribosome, telomerase, Ribonuclease P (RNase P), a heterogeneous ribonucleoprotein particle (hnRNP), or a small nuclear ribonucleoprotein particle (snRNP).

In some embodiments, the protein includes a nuclease. In some embodiments, the nuclease includes a zinc finger nuclease (ZFNs), a transcription-activator like effector nucleases (TALEN), or a Cas protein. In some embodiments, the Cas protein includes Cas2, Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, CjCas9, SpCas9, Cas12, Cas13, Cas14, Cfp1, CasI, CasIB, Cpf1, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu1966, modified versions thereof, or combinations thereof. In some embodiments, the Cas protein is Cas9.

In some embodiments, the negatively charged biological agent includes a nucleic acid and a nuclease. In some embodiments, the negatively charged biological agent includes gRNA and a Cas protein.

In some embodiments, the negatively charged biological agent is bound noncovalently to the polymer.

In another aspect, the invention features a composition including a presently described complex and a liquid carrier.

In another aspect, the invention features a method including contacting a cell with a presently described complex, wherein the biological agent is delivered into the cell.

In another aspect, the invention features a method of identifying polymers for nucleic acid delivery by determining a SHAP value for at least one of polymer repeating units (scaffold RU), cation type, % or type of hydrophobic monomer (e.g., % BET), polymer pKₐ, polymer clogP, polyplex size (Rₕ), formulation (N/P) ratio, and binding strength for a plurality of polymers and selecting monomers for a polymer based on the magnitude and sign of the SHAP value. In some embodiments, the polymer is a polymer of formula (I). In some embodiments, the method further includes synthesizing the polymer and assaying the polymer for expression of a cargo or viability.

In another aspect, the invention features a method of designing and optimizing polymers for nucleic acid delivery by providing a machine learning model trained with data from a plurality of polymers and using Bayesian optimization in the machine learning model to identify new polymers. In some embodiments, the polymer is a polymer of formula (I). In some embodiments, the method further includes synthesizing the polymer and assaying the polymer for expression of a cargo or viability. In some embodiments, data from the assaying is used to retrain the machine learning model, and the method further includes using Bayesian optimization in the retrained model to identify additional polymers. In some embodiments, the design data includes data on scaffold RU, cation type, % of hydrophobic monomer (e.g., % BET), polymer pKₐ, polymer clogP, polyplex size (Rₕ), formulation (N/P) ratio, binding strength, expression level, and/or viability for the plurality of polymers.

### Definitions

To facilitate the understanding of this invention, a number of terms are defined below and throughout the disclosure. Unless otherwise defined, all technical and scientific terms used herein have the same meaning as commonly understood by one of ordinary skill in the art to which this invention belongs. The terminology herein is used to describe specific embodiments of the invention, but their usage does not limit the invention, except as outlined in the claims.

Terms such as “a”, “an,” and “the” are not intended to refer to only a singular entity but include the general class of which a specific example may be used for illustration.

The term “about,” as used herein, refers to a value that is within 10% above or below the value being described.

The term “effective amount,” as used herein refers to the amount that is necessary to result in a physiological change in the cell, organism, or tissue to which it is administered.

The term “individual” or “subject” is an animal, such as a mammal, bird, amphibian, or reptile. Mammals, as used herein, include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition,” as used herein, refers to a preparation which is in such form as to permit the biological activity of an active ingredient contained therein to be effective, and which contains no additional components which are unacceptably toxic to a subject to which the formulation would be administered.

The term “pharmaceutically acceptable carrier,” as used herein, refers to an ingredient in a pharmaceutical composition, other than an active ingredient, which is nontoxic to a subject. A pharmaceutically acceptable carrier includes, but is not limited to, a buffer, excipient, stabilizer, or preservative.

The term “therapeutically effective amount,” as used herein, e.g., of a pharmaceutical composition, refers to an amount effective, at dosages and for periods of time necessary, to achieve the desired therapeutic or prophylactic result. A therapeutically effective amount of an agent for example eliminates, decreases, delays, minimizes, reduces, or prevents adverse effects of a disease.

The term “alkenyl,” as used herein, refers to an acyclic straight or branched chain monovalent hydrocarbon group containing one or more double bonds, no triple bonds, and from 2 to 12 (e.g., 2 to 6) carbons, unless otherwise specified. Alkenyl groups may be substituted or unsubstituted. Exemplary substituents include alkoxy, alkylthio, alkynyl, amido, amino, carbonate, carboxyl, cyano, epoxy, halo, heterocyclyl, hydroxyl, oxo, and thiol.

The term “alkyl,” as used herein, refers to an acyclic straight or branched chain, saturated, monovalent hydrocarbon group having from 1 to 12 carbons (e.g., 1 to 6), unless otherwise specified.

Alkyl groups may be substituted or unsubstituted. Exemplary substituents include alkoxy, alkylthio, amido, amino, carbonate, carboxyl, cyano, epoxy, halo, heterocyclyl, hydroxyl, oxo, and thiol. An alkyl may be substituted with an oxo to form an aldehyde or ketone.

The term “alkynyl,” as used herein, refers a straight or branched monovalent hydrocarbon group containing one or more triple bonds and from 2 to 12 (e.g., 2 to 6) carbons, unless otherwise specified. Alkynyl groups may be unsubstituted or substituted as alkenyl groups.

The term “alkoxy,” as used herein, refers to a group of the formula RO—, wherein R is an alkyl group as defined herein. Alkoxy groups may be unsubstituted or substituted as alkyl groups. An alkoxy may be substituted with an oxo group to form an ester. Three alkoxy groups may be bound to the same carbon to form an orthoester.

The term “alkylthio,” as used herein, refers to a group of the formula RS—, wherein R is an alkyl group as defined herein. Alkylthio groups may be unsubstituted or substituted as alkyl groups.

The term “alkylene,” as used herein, refers to a divalent group obtained by removing a hydrogen from a carbon atom of an alkyl group. Alkylene groups may be unsubstituted or substituted as alkyl groups.

The term “amido,” as used herein, refers to a group of the formula —C(═O)NR′R″, where each of R′ and R″ are independently H or alkyl.

The term “amino,” as used herein, refers to a group of formula —NR′R″ or —NR′R″R′″⁺, where each of R′, R″, and R′″ are independently H or alkyl.

The term “aryl,” as used herein, refers to any monocyclic or fused ring bicyclic or multicyclic system containing only carbon atoms in the ring(s), which has the characteristics of aromaticity in terms of electron distribution throughout the ring system, e.g., phenyl, naphthyl, or phenanthryl. An aryl group may have, e.g., six to sixteen carbons (e.g., six carbons, ten carbons, thirteen carbons, fourteen carbons, or sixteen carbons). Aryl groups may be unsubstituted or substituted. Exemplary substituents include alkyl, alkoxy, alkylthio, amido, amino, aryl, carbonate, carboxyl, cyano, epoxy, halo, heterocyclyl, hydroxyl, and thiol.

The term “carbonate,” as used herein, refers to a group of the formula —OC(═O)OR, wherein R is H or alkyl.

The term “carboxyl,” as used herein, refers to a group of the formula —(C═O)OH.

The term “cyano,” as used herein, refers to —C≡N.

The term “epoxy,” as used herein, refers to >O, where the oxygen is bound to adjacent carbon atoms.

The term “halide,” as used herein, refers to a F, Cl, Br, or I anion.

The term “halo,” as used herein, refers to a F, Cl, Br, or I radical.

The term “heterocyclyl,” as used herein, represents a monovalent, monocyclic or fused ring bicyclic or multicyclic system having at least one heteroatom as a ring atom. For example, a heterocyclyl group may have, e.g., one to fifteen carbon ring atoms (e.g., a C₁-C₂, C₁-C₃, C₁-C₄, C₁-C₅, C₁-C₆, C₁-C₇, C₁-C₈, C₁-C₉, C₁-C₁₀, C₁-C₁₁, C₁-C₁₂, C₁-C₁₃, C₁-C₁₄, or C₁-C₁₅ heterocyclyl) and one or more (e.g., one, two, three, four, or five) ring heteroatoms independently selected from the group consisting of nitrogen, oxygen, and sulfur. Heterocyclyl groups may or may not include a ring that is aromatic. Heterocyclyl groups may be unsubstituted or substituted. In preferred embodiments of the invention, a heterocyclyl group is a 3- to 8-membered ring, a 3- to 6-membered ring, a 4- to 6-membered ring, a 5-membered ring, or a 6-membered ring. Exemplary 5-membered heterocyclyl groups may have zero to two double bonds, and exemplary 6-membered heterocyclyl groups may have zero to three double bonds. The term “basic nitrogen containing heterocycle,” as used herein, refers to a heterocyclyl group having at least one ring nitrogen (e.g., 1 to 4 nitrogen atoms, e.g., 1 or 2) that can accept a proton from solution, e.g., imidazolyl or benzimidazolyl. Heterocyclyl groups may be substituted or unsubstituted. Aryl groups may be unsubstituted or substituted. Exemplary substituents include alkyl, alkoxy, alkylthio, amido, amino, aryl, carbonate, carboxyl, cyano, epoxy, halo, heterocyclyl, hydroxyl, and thiol.

The term “oxo,” as used herein, refers to ═O.

The term “thiol,” as used herein, refers to —SH.

### BRIEF DESCRIPTION OF THE DRAWINGS

The following detailed description of the embodiments of the invention will be better understood when read in conjunction with the appended drawings. For the purpose of illustrating the invention, there are shown in the drawing embodiments, which are presently exemplified. It should be understood, however, that the invention is not limited to the precise arrangement and instrumentalities of the embodiments shown in the drawings.

FIG. 1 shows polymers generated through the present high-throughput polymer scaffold approach assembled into complexes with biological agents, such as pDNA and RNP. FIG. 1 further shows that the polymer formulations went through a loop machine learning approach using Bayesian optimization to identify further polymers for exploration. Functional and physical characteristics linked the polymer feature importance to the expression and viability outputs to identify structure-property relationships. FIG. 1 also shows that polymers of interest were evaluated for efficient delivery of pDNA into mice.

FIG. 2 shows (top) representative chemical schematic of the poly(allylmethacrylamide) (pAMAm) polymer scaffold undergoing a stepwise thiol-ene post polymerization modification of the medium backbone with 20% benzimidazole ethanethiol (BET) and then further split into three to be saturated with the remaining cations (Cys, Cap, DiE). The functional amines have a range of charge state (pKa) and hydrophobicity (clogP) characteristics. FIG. 2 further shows (bottom) a visual representation of the 36 polymers in the library showing the repeat units in the parent backbone (N=90, 190, 250) and the range in BET incorporation within each polymer set.

FIG. 3A shows a schematic depicting a generalized approach to how the polymer library was screened to bind and deliver pDNA and CRISPR RNP into HEK293T cells and to detect their fluorescent output of GFP or mCherry. FIG. 3B shows a top expressing polymer of each cation in comparison to the pDNA and JetPEI controls. FIG. 3B shows the respective viability of each system. FIG. 3C shows heat maps visualizing the % cells with expression, cell viability, binding, and complex size (Rh). The diagrams in FIG. 3C show the polymer library with pDNA (top row, N/P 10) and RNP (bottom row, N/P 5).

FIG. 4A shows a looped machine learning approach by tuning N/P ratio based on polymer characteristics to affect delivery efficiency and cell viability through Bayesian optimization. FIG. 4B shows sequential optimization data from the 3 rounds of experimental expression of GFP (top) and mCherry (bottom). FIG. 4C shows parallel coordinate plots showing the polymer compositions and the effective expression of GFP (top) and mCherry (bottom) through three rounds.

FIG. 5A shows Shapley Additive Explanations (SHAP) values related to expression and viability in relation to pDNA transfection. Higher SHAP values are correlated to higher impact on the output variable. The feature value colorbar corresponds to the normalized value of the feature of interest. FIG. 5B shows SHAP values related to expression and viability in relation to RNP transfection. FIG. 5C shows SHAP dependency plots values across two variables relating to expression.

FIG. 6A shows kinetic hydrodynamic tail vein study showing the average radiance (p/s/cm²/sr) emitted by mice over a 20-day study after delivery of a luciferase expressing pDNA (n=3). FIG. 6B shows a table displaying average radiance values (p/s/cm²/sr) and a decay rate displayed first-order kinetics of triplicate mice of mice days 1, 2, 3, and 6. FIG. 6C shows images of mice in triplicate showing heat map expression on days 1, 2, 3, and 6 post injection. FIG. 6D shows average weight of mice per sample group over the 20 days. Day 0 is injection day.

FIG. 7A shows dye exclusion data showing the amount of fluorescence of PicoGreen after the addition of the short length backbone functionalized polymers at an N/P ratio of 10 and 20. FIG. 7B shows dye exclusion data showing the amount of fluorescence of PicoGreen after the addition of the medium length backbone functionalized polymers at an N/P ratio of 10 and 20. FIG. 7C shows dye exclusion data showing the amount of fluorescence of PicoGreen after the addition of the long length backbone functionalized polymers at an N/P ratio of 10 and 20.

FIG. 8A shows viability data showing the normalized transmittance in a CCK8 assay when the short backbone polymers delivered RNP into HEK293T cells at an N/P ratio of 10 and 20. FIG. 8B shows viability data showing the normalized transmittance in a CCK8 assay when the medium backbone polymers delivered RNP into HEK293T cells at an N/P ratio of 10 and 20. FIG. 8C shows viability data showing the normalized transmittance in a CCK8 assay when the long backbone polymers delivered RNP into HEK293T cells at an N/P ratio of 10 and 20.

### DETAILED DESCRIPTION OF THE INVENTION

The disclosure provides polymeric delivery vehicles for the delivery of biological agents.

The present polymers may be synthesized from poly(pentafluorophenyl methacrylate) (pPFPMA). The polymer pPFPMA is advantageous in that it offers the ability to synthetically tailor the polymer through post-polymerization modification. Herein, PFPMA has been shown to be able to be polymerized using the reversible addition-fragmentation chain transfer (RAFT) mechanism to control degree of polymerization and dispersity of the backbone. The pentafluorophenol group is labile and causes the activated ester to be modified with synthetic ease by reacting with a variety of functional amines through an amidation reaction. In some embodiments, the pPFPMA backbone was modified with allylamine, resulting in poly(allylmethacrylamide) (pAMAm), a polymer backbone decorated with pendent alkenes that are susceptible for a subsequent step of post-polymerization with thiol-ene click chemistry. In some embodiments, the pAMAm polymer scaffold may be synthetically modified with heterocycles and amines, e.g., benzimidazole ethanethiol (BET), cysteamine (Cys), captamine (Cap), and 2-(diethylamino) ethanethiol (DiE) in various stoichiometries to develop a plurality of polymers.

The present polymeric delivery systems harness the use of cationic polymers as delivery systems to take advantage of nucleic acid backbones having a negative charge due to the phosphate groups along each nucleotide. The present polymeric delivery vehicles may condense biological agents, such as CRISPR payloads (for example, mRNA, pDNA or ribonucleoproteins (RNP)), which can vary widely in their lengths, topologies, physical characteristics and biological mechanisms, into discrete nanosized polyelectrolyte complexes. Upon administration, the complexes may navigate both extracellular barriers such as serum DNAases (or RNAases) and reticuloendothelial system clearance, as well as intracellular barriers such as endosomal interrogation and lysosomal degradation. Finally, the biological agent may be released within the spatiotemporal window that is optimal for payload translocation to the nucleus, where the biological agent can undergo further processing and realization of targeted edits. In addition to meeting high standards for safety, efficiency and cost-effectiveness, synthetic delivery systems may minimize immune activation and cellular toxicity.

In general, the present disclosure is directed to polymers that may be associated with at least one biological agent payload such as pDNA, RNP, and the like. The complexes may be internalized by a cell via various endocytic routes, the biological agent may be released inside the cell, and it subsequently may enter the cell nucleus to alter gene expression. The polymers disclosed herein thus provide a polymeric scaffold that provides a well-defined host configured to bind with biological macromolecular agents and facilitate intracellular delivery thereof. The polymers have physiochemical properties such as, for example, composition, molecular weight, ζ-potential, pKa, complex diameter, nucleic acid condensation, and combinations thereof selected for efficient nucleic acid payload delivery using, for example, a CRISPR/Cas9 delivery process. The polymers and related complexes may also have good gene editing efficiency, cellular internalization, cytotoxicity, and combinations thereof.

### Polymers

The present invention relates to a polymer of formula (I):

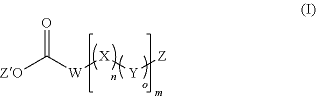

wherein W is optionally substituted alkylene; each X is independently

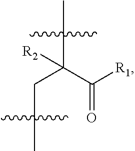

wherein R₁ is —O—C₆F₅ or —NR′R″, wherein R′ and R″ are each independently H, alkenyl, or substituted alkylthio alkyl, and
wherein R₂ is H or CH₃; each Y is independently

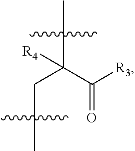

wherein R₃ is —NR′R″, wherein R′ and R″ are each independently H, alkenyl, or substituted alkylthio alkyl, and wherein R₄ is H or CH₃; Z is hydrogen, alkyl, thiol, or

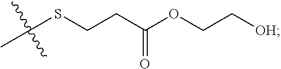 [SMILES: *SCCC(=O)OCCO]

Z′ is H, or alkyl; m is from 1 to 1000; each of n and o represents a fraction of m, and the sum of n and o is 1; or an ion or salt thereof.

In particular embodiments, at least one X or Y is

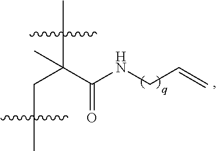

wherein q is 1 to 4.

In particular embodiments, at least one X or Y is

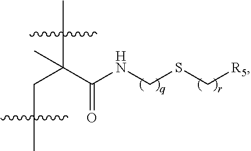

wherein R₅ is NRₐR_{b}, where Rₐ and R_{b} are independently H or alkyl, and q and r are independently 1 to 6.

In particular embodiments, at least one X or Y is

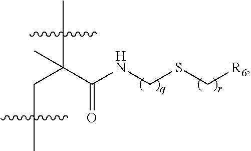

wherein R₆ is a basic nitrogen containing heterocycle, and q and r are independently 1 to 6.

In some embodiments of formula (I), the basic nitrogen containing heterocycle is

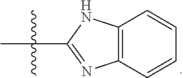 [SMILES: *c1nc2ccccc2[nH]1]

In some embodiments, W is substituted with cyano.

The present invention further provides to a compound of formula (II):

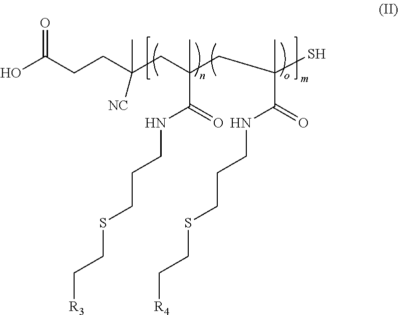

wherein, R₃ is NH₂, N(CH₃)₂, or N(C₂H₅)₂; R₄ is a basic nitrogen containing heterocycle; m is from 1 to 1000; each of n and o represents a fraction of m, and the sum of n and o is 1; or a salt or ion thereof.

In some embodiments of formula (II), the basic nitrogen containing heterocycle is

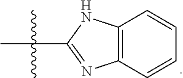 [SMILES: *c1nc2ccccc2[nH]1]

In particular embodiments, the compound of formula (II) is selected from:

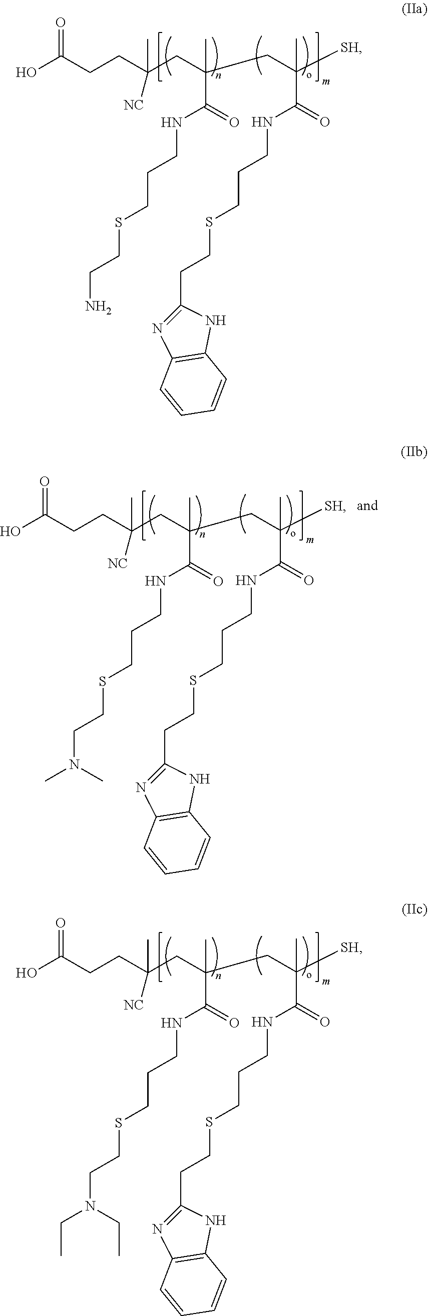

wherein m is from 1 to 1000; each of n and o represents a fraction of m, and the sum of n and o is 1; or a salt or ion thereof.

In formulas (I), (II), (IIa), (IIb), and (IIc), m may be from about 1 to about 1000, e.g., from 1 to 2, 1 to 5, 1 to 10, 1 to 20, 1 to 25, 1 to 30, 1 to 40, 1 to 50, 1 to 60, 1 to 70, 1 to 75, 1 to 80, 1 to 90, 1 to 100, 1 to 125, 1 to 150, 1 to 175, 1 to 200, 1 to 250, 1 to 300, 1 to 400, 1 to 500, 1 to 600, 1 to 700, 1 to 800, 1 to 900, 2 to 5, 2 to 10, 2 to 25, 2 to 50, 5 to 10, 5 to 15, 5 to 20, 5 to 25, 5 to 30, 5 to 35, 5 to 40, 5 to 45, 5 to 50, 5 to 55, 5 to 60, 5 to 70, 5 to 75, 5 to 80, 5 to 90, 5 to 100, 10 to 20, 10 to 25, 10 to 30, 10 to 40, 10 to 50, 10 to 60, 10 to 70, 10 to 80, 10 to 90, 10 to 100, 15 to 20, 15 to 25, 15 to 30, 15 to 45, 16 to 24, 17 to 23, 18 to 22, 19 to 21, 20 to 30, 20 to 40, 20 to 50, 20 to 60, 20 to 70, 20 to 75, 20 to 80, 20 to 90, 20 to 100, 25 to 30, 25 to 35, 25 to 40, 25 to 50, 25 to 75, 25 to 100, 25 to 125, 26 to 34, 27 to 33, 28 to 32, 29 to 31, 30 to 35, 30 to 40, 30 to 45, 30 to 50, 30 to 60, 30 to 70, 30 to 80, 30 to 90, 30 to 100, 35 to 65, 40 to 50, 40 to 60, 40 to 70, 40 to 80, 40 to 90, 40 to 100, 50 to 60, 50 to 70, 50 to 80, 50 to 90, 50 to 100, 50 to 125, 50 to 150, 50 to 175, 50 to 200, 50 to 250, 50 to 300, 50 to 350, 50 to 400, 50 to 450, 50 to 500, 50 to 550, 50 to 600, 50 to 700, 50 to 800, 50 to 900, 50 to 1000, 60 to 100, 60 to 140, 70 to 100, 70 to 130, 80 to 100, 80 to 120, 90 to 110, 100 to 125, 100 to 150, 100 to 200, 100 to 300, 100 to 500, 100 to 750, 100 to 1000, 150 to 200, 150 to 250, 200 to 300, 200 to 500, 200 to 700, 200 to 1000, 250 to 500, 250 to 750, 250 to 1000, 300 to 500, 300 to 700, 400 to 500, 400 to 600, 500 to 600, 500 to 750, 500 to 800, 500 to 1000, 750 to 1000, 800 to 900, 800 to 1000, or 900 to 1000, e.g., is about 10, 25, 50, 75, 90, 100, 125, 150, 190, 200, 225, 250, 300, 350, 400, 450, or 500.

In formulas (I), (II), (IIa), (IIb), and (IIc), n may be from about 0 to about 1, e.g., from 0 to 0.05, 0 to 0.1, 0 to 0.15, 0 to 0.2, 0 to 0.25, 0 to 0.3, 0 to 0.35, 0 to 0.4, 0 to 0.45, 0 to 0.5, 0 to 0.55, 0 to 0.6, 0 to 0.65, 0.7, 0 to 0.75, 0 to 0.8, 0 to 0.85, 0 to 0.9, 0 to 0.95, 0.05 to 1, 0.1 to 1, 0.15 to 1, 0.2 to 1, 0.25 to 1, 0.3 to 1, 0.35 to 1, 0.4 to 1, 0.45 to 1, 0.5 to 0.65, 0.5 to 1, 0.55 to 0.6, 0.55 to 1, 0.6 to 1, 0.65 to 1, 0.7 to 0.9, 0.7 to 1, 0.75 to 0.83, 0.75 to 1, 0.8 to 0.95, 0.8 to 1, 0.85-0.9, 0.85 to 1, 0.9 to 1, or 0.95 to 1, e.g., is about 0.1, 0.2, 0.3, 0.4, 0.5, 0.55, 0.6, 0.7, 0.75, 0.8, 0.85, 0.9, or 1.

In formulas (I), (II), (IIa), (IIb), and (IIc), o may be from about 0 to about 1, e.g., from 0 to 0.05, 0 to 0.1, 0 to 0.15, 0 to 0.2, 0 to 0.25, 0 to 0.3, 0 to 0.35, 0 to 0.4, 0 to 0.45, 0 to 0.5, 0 to 0.55, 0 to 0.6, 0 to 0.65, 0.7, 0 to 0.75, 0 to 0.8, 0 to 0.85, 0 to 0.9, 0 to 0.95, 0.05 to 1, 0.05 to 0.2, 0.1 to 0.15, 0.1 to 1, 0.1 to 0.3, 0.15 to 1, 0.17 to 0.25, 0.2 to 1, 0.25 to 1, 0.3 to 1, 0.35 to 1, 0.35 to 0.5, 0.4 to 0.45, 0.4 to 1, 0.45 to 1, 0.5 to 1, 0.55 to 1, 0.6 to 1, 0.65 to 1, 0.7 to 1, 0.75 to 1, 0.8 to 1, 0.85 to 1, 0.9 to 1, or 0.95 to 1, e.g., is about 0.1, 0.15, 0.2, 0.25, 0.3, 0.4, 0.45, 0.5, 0.6, 0.7, 0.8, 0.9, or 1.

### Complexes

The polymers described herein may bind with a biological agent. The biological agent may be bound to the polymer in a variety of methods. In some embodiments, the biological agent is bound noncovalently to the polymer, e.g., electrostatically to the polymer. In some embodiments, the polymer may be complexed with the biological agent. In some embodiments, the polymer may be condensed with the biological agent. Without wishing to be bound to theory, the polymers described herein, including those bound, complexed, or condensed to negatively charged biological agents, may be able to evade the immune system by mimicking bacteria-like, less-foreign morphologies, thereby showing great promise as a multiplexable system.

The present disclosure provides a complex including a polymer of formula (I) or (III), and a biological agent. In some embodiments, the biological agent is a negatively charged biological agent. The biological agent may include a therapeutic agent. The biological agent may include a nucleic acid, a peptide, a protein, or a small molecule.

The biological agent may include a small molecule. Small molecules are compounds with low molecular weight that are capable of modulating biochemical processes to diagnose, treat, or prevent diseases. The present polymeric delivery systems may be used to transport small-molecule therapeutics to cells.

The biological agent may include a nucleic acid. Without wishing to be bound to theory, when negatively charged biological agents (e.g., nucleic acids such as RNA or other small molecule therapeutics) are covalently attached to the bottlebrush architecture, the circulation time of the cargo may be increased in vivo. The nucleic acid may be DNA, RNA, or chimeric. In some embodiments, the nucleic acid includes gRNA, mRNA (e.g., that encodes for proteins (fluorescent or therapeutic), tmRNA, tRNA, rRNA, siRNA, shRNA, PNA, ssRNA, dsRNA, pDNA (e.g., that encodes for proteins (fluorescent or therapeutic), ssDNA, dsDNA, a DNA:RNA hybrid molecule, DNA editing templates, miRNA, an artificial chromosome, oligo nucleotide, a nucleic acid encoding a nuclease, cDNA, a PCR product, a restriction fragment, a ribozyme, an antisense construct, or a combination thereof. Nucleic acids may include modification to the sugar, backbone, and/or base and may include synthetic or non-canonical bases.

A gRNA includes an engineered, non-naturally occurring Clustered Regularly Interspaced Short Palindromic Repeats (CRISPR) gRNA that hybridizes with a target nucleic acid sequence of interest.

In various embodiments, DNA editing templates include an exogenous strand of DNA that bears homology arms to a section of genomic DNA that has been cut by a nuclease (for example, Cas9, TALEN or zinc finger) along with an intervening sequence between these homology arms that differs with the natural segment of genomic DNA that has been cut. This intervening segment serves as the template for repair of the cut genomic DNA and, in so doing, the cell corrects its own DNA to match that of the DNA template. The DNA template may be included in a single DNA expression vector that also encodes the nuclease.

siRNAs refer to a double-stranded interfering RNA. In addition to siRNA molecules, other interfering RNA molecules and RNA-like molecules may be used. Examples of other interfering RNA molecules that may to inhibit target biomolecules include, but are not limited to, short hairpin RNAs (shRNAs), single-stranded siRNAs, microRNAs (miRNAs), piwiRNA, Dicer-substrate 27-mer duplexes, and variants thereof containing one or more chemically modified nucleotides, one or more non-nucleotides, one or more deoxyribonucleotides, and/or one or more non-phosphodiester linkages. Typically, all RNA or RNA-like molecules that may interact with transcripts RISC complexes and participate in RISC-related changes in gene expression may be referred to as interfering RNAs or “interfering RNA molecules.

Suitable interfering RNAs may readily be produced based on the well-known nucleotide sequences of target biomolecules. In various embodiments interfering RNAs that inhibit target biomolecules may include partially purified RNA, substantially pure RNA, synthetic RNA, recombinant produced RNA, as well as altered RNA that differs from naturally occurring RNA by the addition, deletion, substitution, and/or alteration of one or more nucleotides. Such alterations may include, for example, addition of non-nucleotide material, such as to the end(s) of the interfering RNAs or to one or more internal nucleotides of the interfering RNAs, including modifications that make the interfering RNAs resistant to nuclease digestion. Such alterations result in sequences that are generally at least about 80%, or more, or even 100% identical to the sequence of the target biomolecule. When the gene to be down regulated is in a family of highly conserved genes, the sequence of the duplex region may be chosen with the aid of sequence comparison to target only the desired gene. On the other hand, if there is sufficient identity among a family of homologous genes within an organism, a duplex region may be designed that would down regulate a plurality of genes simultaneously.

The N/P ratio of a complex is the ratio of positively-chargeable polymer amine (N=nitrogen) groups to negatively-charged nucleic acid phosphate (P) groups. The N/P character of a polymer/nucleic acid complex may influence complex properties, such as its net surface charge, size, and stability of the complex. The N/P ratio of the present complexes may be from 0 to 10, e.g., from 0 to 1, 0 to 2, 0 to 3, 0 to 4, 0 to 5, 0 to 6, 0 to 7, 0 to 8, 0 to 9, 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, 9 to 10, or about 0.5, about 1, about 2, about 3, about 4, about 5, about 6, about 7, about 8, about 9, or about 10.

Alternatively, or in addition, the biological agent may include a peptide. In various embodiments, peptide fragments include two or more amino acids covalently linked by at least one amide bond. For example, in some embodiments the peptide fragments may include pDNA encoded fluorescent or therapeutic peptides.

Alternatively, or in addition, the biological agent may include a protein. In some embodiments, the protein may include an antibody. The antibody may be a monoclonal antibody.

In some embodiments, the protein includes a ribonucleoprotein. The ribonucleoprotein may include a ribosome, telomerase, ribonuclease P (RNase P), a heterogeneous ribonucleoprotein particle (hnRNP), or a small nuclear ribonucleoprotein particle (snRNP). sgRNA may be chemically modified to improve stability and prevent intracellular degradation.

In some embodiments, the protein may include a nuclease. The nuclease may include a zinc finger nuclease (ZFNs), a transcription-activator like effector nucleases (TALEN), or a Cas protein. The Cas protein may be a Cas2, Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, CjCas9, SpCas9, Cas12, Cas13, Cas14, CfpI, CasI, CasIB, Cpf1, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu1966, modified versions thereof, or combinations thereof. In some embodiments, the Cas protein is Cas9.

In some embodiments, the biological agent includes a nucleic acid and a nuclease. The negatively charged biological agent may be gRNA and a Cas protein, as in the CRISPR-Cas system. The CRISPR-Cas system is useful for precise editing of genomic nucleic acids (e.g., for creating null mutations). For example, a composition containing only the guide RNA can be administered to an animal or cells transgenic for the Cas9 enzyme. Similar strategies may be used (e.g., zinc finger, transcription activator-like effectors (TALEs) or homing meganucleases).

The CRISPR-Cas system is known in the art for deleting, modifying genome sequences or incorporating transgenes. Transgene refers to any nucleotide sequence, particularly a DNA sequence, that is integrated into one or more chromosomes of a host cell by human intervention, such as by the methods of the present invention. For example, a transgene can be an RNA coding region or a gene of interest, or a nucleotide sequence, preferably a DNA sequence, that is used to mark the chromosome where it has integrated or may indicate a position where nucleic acid editing, such as by the CRISPR-CAS system, may occur. In this situation, the transgene does not have to include a gene that encodes a protein that may be expressed. CRISPR-Cas genome editing has rapidly emerged as a multi-faceted technology to enable gene insertion, deletion, activation, suppression, and even single base editing of target genes within the nucleus of any cell. This highly efficient and facile technique has broad utility from white biotechnology and agriculture to biomedical research, pharmaceutics, and regenerative medicine.

Currently, the CRISPR/Cas9 system can be delivered in vitro, ex vivo, and in vivo in three different payload forms: i) pDNA that encodes Cas9 protein and/or sgRNA ii) mRNA that encodes for Cas9 nuclease and a separate sgRNA, or iii) a ribonucleoprotein (RNP) that consists of recombinant Cas9 protein precomplexed directly with a sgRNA.

CRISPR-Cas9 pDNA needs to enter the cellular nucleus to express, and consistent expression produces an overabundance of Cas9 protein, which can lead to increased off-target editing and mutagenesis. Researchers have utilized the CRISPR/Cas9 system in mRNA form to circumvent the barrier of nuclear entry, which has been reported with polymer-based nanoparticles. However, sgRNA often needs to be delivered separately, presenting challenges in trafficking kinetics of different payloads.

Direct delivery of CRISPR/Cas9 ribonucleoprotein (RNP) has several benefits, including precision in endonuclease dosing and potential to avoid uncontrolled integration of the transgene into the cellular genome.

### Formulations

In another aspect, the present disclosure is directed to compositions including the polymer complexes described above which have been dispersed in a solution. In some embodiments, the composition includes a plurality of polymers described herein which have been dispersed in a solution, e.g., a mixture of 2, 3, 4, 5, or more. When multiple polymers are employed, they may differ in the fraction of monomeric units, i.e., in o and n, or they may differ in the chemical structure of the monomeric units. In some embodiments, the complexes may be added to a liquid carrier and stored in liquid form until needed, or alternatively may be dried and introduced into and dispersed in the liquid carrier prior to use, e.g., administration to a subject.

In some embodiments the liquid carrier is a pharmaceutically acceptable carrier. Some non-limiting examples of materials which can serve as pharmaceutically-acceptable carriers include pyrogen-free water; isotonic saline; Ringer's solution; ethyl alcohol; pH buffered solutions. The formulation may include solvents, dispersing medium (containing, e.g., water, cell culture medium, buffers (e.g., phosphate buffered saline), polyol (for example, glycerol, propylene glycol, or liquid polyethylene glycol), wetting agents, emulsifying agents, coloring agents, release agents, coating agents, sweetening agents, flavoring agents, perfuming agents, pH buffering agents, gelling or viscosity enhancing additives, preservatives, colors, preservatives, or antioxidants.

The compounds of formula (I) or (II) may be ionized in dry or liquid formulation. For example, amine or basic nitrogen groups may be protonated. The compounds of formula (I) or (II) may be in salt form with one or more anions, e.g., acetate, ascorbate, benzoate, bicarbonate, bisulfate, carbonate, cholate, citrate, dihydrogen citrate, glycocholate, halide (such as F, Cl, Br, or I), hydrogen citrate, hydroxide, mandelate, methanesulfonate, nitrate, oxalate, p-toluenesulfonate, persulfate, phosphate, lactate, succinate, sulfate, sulfite, tartrate, taurocholate, or trifluoroacetate.

### Methods of Use

The present disclosure provides methods of contacting a cell with the herein-described complexes, wherein the biological agent is delivered into the cell.

For example, after a composition including the polymer and bound biological agent are contacted with a cell, the complexes internalize into the cell, and the biological agent disassociate partially or completely from the polymeric carriers.

The formulations described herein can be delivered to a cell or an organism via any administration mode known to a skilled practitioner. For example, the formulations described herein can be delivered via administration routes such as, but not limited to, in vitro, oral, intravenous, intramuscular, intraperitoneal, intradermal, and subcutaneous. In some embodiments, the compositions described herein are in a form that is suitable for injection. In other embodiments, the formulations described herein are formulated for oral administration. In various embodiments, for in vivo administration a delivery device can be used to facilitate the administration of any composition described herein to a subject, e.g., a syringe, a dry powder injector, a nasal spray, a nebulizer, or an implant such as a microchip, e.g., for sustained release or controlled release of any formulation described herein.

In various embodiments, the compositions may be administered to a cell in vitro by removing a cell from a subject, culturing the cells, applying to the cells a composition including polymer vehicles and bonded biological agent to deliver a therapeutic amount of the biological agent into at least a portion of the cells, and optionally re-introducing the cell to the subject.

In another embodiment, a tissue cell therapy technique may be used in which a tissue sample is removed from a subject, a composition including a polymer and a bonded biological agent is applied to the tissue to deliver a therapeutic amount of the biological agent to modify a selected cell or region of the tissue, and the modified tissue is transplanted into the subject.

### Machine Learning

The present disclosure provides methods of characterizing, optimizing and designing polymers, e.g., for nucleic acid delivery, and methods of identifying formulations, e.g., for in vitro delivery. The methods employ structure-property relationships modeling (see, e.g., JACS Au 2022, 2 (2), 428-442. And ACS Nano 2020, 14 (12), 17626-17639). A regression model may be used to predict the expression and viability for each cargo based on the following polyplex features: scaffold RU, cation type, % hydrophobic monomer (e.g., % BET), polymer pKₐ, polymer clogP, polyplex size (Rₕ), formulation (N/P) ratio, and/or binding strength. The model and its hyperparameters may be chosen based on satisfactory performance on polymer-grouped cross validation. This trained model may be employed for interpretability via Shapley Adaptive Explanations (SHAP) (see, e.g., Proc. 31st Int. Conf. Neural Inf. Process. Syst. 2017, pp 4768-4777). SHAP provides local and global explanations for the relative importance of each feature by learning a local linear model with game-theoretic constraints. In particular, the method can apply the TreeSHAP algorithm and take the mean absolute SHAP value across all data points as our feature importance (radar plots) and analyze trends in local SHAP values.

Data from polymers and their expression efficacy and viability can be used to train a probabilistic or Bayesian machine learning model, such as a Gaussian process regression model. Once trained, Bayesian optimization (BO) may be employed to identify new polymers to maximize expression efficacy. BO is a machine learning approach that employs sequential learning to efficiently explore combinatorial design spaces. With our current scaffolds, when considering multifactorial optimization of polymer pKₐ, polymer clogP, % or type of hydrophobic monomer (e.g., % BET), scaffold RU, and formulation N/P ratio, a uniform or quasi-random sampling (e.g., using design of experiments) of the variable space may hide potentially high performing polymer sets and may require a large number of samples to identify the best performing polymers. Bayesian optimization performs this optimization efficiently. Also, the BO method may be designed to maximize expression while maintaining viability (or other design constraints) above a set threshold. For example, once candidate polymers are suggested by the BO optimization algorithm, an auxiliary model may be employed to predict viability and avoid toxic polymer designs.

As the polymers are optimized in a mixed space of continuous (N/P) and discrete variables (e.g., cation choice), the method may employ a mixed space kernel in the machine learning model. To improve predictive performance of the probabilistic model, the method may map the cation monomer information to an encoded representation based on its extended connectivity fingerprint (J. Chem. Inf. Model. 2010, 50 (5), 742-754), appended to calculated polymer clogP and polymer pKₐ values. This mapping leads to a model that better encodes similarities across monomers compared to purely categorical choices and is thus easier to optimize using Gaussian process regression. The method may employ one or more, e.g., three rounds, of Bayesian optimization for each cargo. In each case, a batch of polymers can be synthesized, and the in vitro or in vivo performance measured. The Gaussian process model may be updated with the expression results to propose a batch of new samples by using an expected improvement acquisition function and Thomson sampling (Proc. IEEE 2016, 104 (1), 148-175). This batch of new samples corresponds to samples that are expected to have good performance by being similar to explored areas of the design space (exploitation) or samples in areas of the design space that have high expected variability (exploration). For each round, the method may verify that the suggested samples satisfied basic cellular toxicity requirements by fitting the viability output with an auxiliary machine learning model, e.g. a random forest model.

### EXAMPLES

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Example 1: Polymer Synthesis and Functionalization

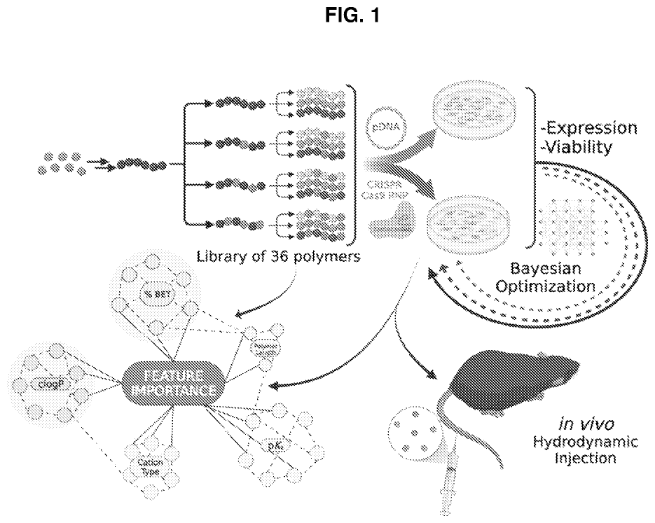

This example demonstrates the use of polymer scaffolds to produce a well-defined library of cationic polymers designed to study structure property relationships directed towards effective gene delivery, as shown in FIG. 1.

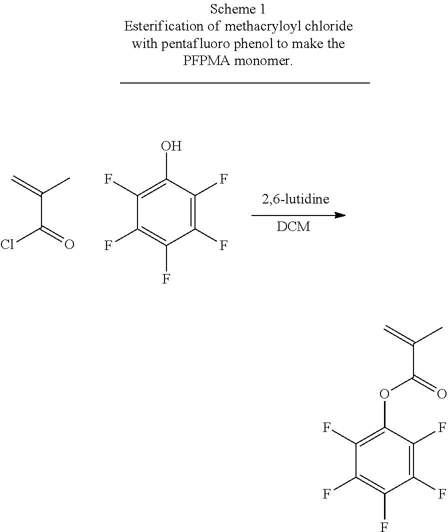

An activated ester was synthesized through an esterification of methacryloyl chloride with pentafluoro phenol to produce PFPMMA (36 g, 80%). Pentafluorophenol (33 g, 0.179 mol) was dissolved in 100 mL anhydrous dichloromethane (DCM) and allowed to cool in an ice bath for 30 min. After being cooled to 0° C., 2,6-lutidine (20.8 mL, 0.179 mol) was added and the mixture was stirred over ice, followed by an additional 50 mL of anhydrous DCM. Methacryloyl chloride (21 mL, 0.215 mol) was mixed with 50 mL anhydrous DCM and was slowly added to the round-bottom flask via an addition funnel over a span of 1 h. When the addition was complete, a white solid crashed out of solution, 2,6-lutidine-hydrochloric acid (HCl). The reaction was stirred overnight at room temperature. The reaction mixture was filtered to remove the white salt, and then an aqueous work up was completed with deionized (DI) water (3×200 mL) and brine (1×150 mL). The aqueous solution was further extracted with DCM (1×200 mL). The reaction mixture was dried over magnesium sulfate, filtered, and then concentrated in vacuo. The slight yellow liquid product was further purified via vacuum distillation 40° C. at 100 mTorr to yield a colorless liquid (36 g, 80%). ¹H nuclear magnetic resonance (NMR) (400 MHz, Chloroform-d) δ 6.45 (s, 1H), δ 5.91 (s, 1H), δ 2.09 (s, 3H). ¹⁹F NMR (400 MHz, Chloroform-d) δ −152.8 (d, 2F), −158.2 (t, 1F), −162.5 (d, 2F).

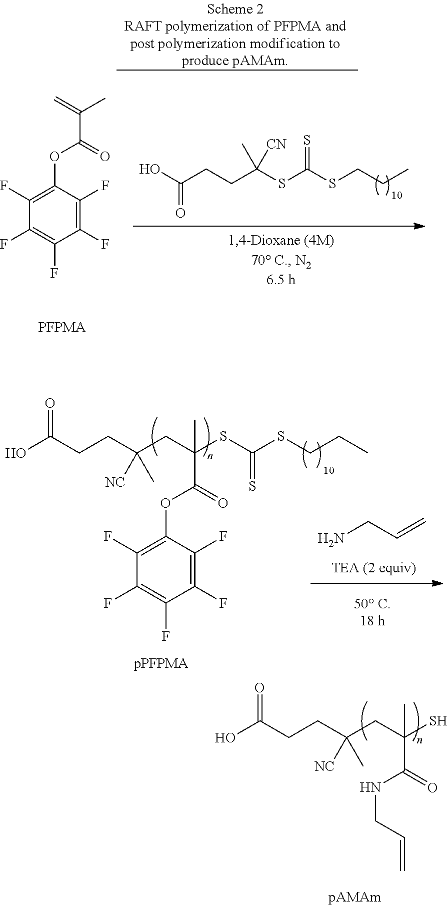

The PFPMA underwent RAFT polymerization in order to synthesize three well-defined polymer lengths that were to be further modified. The three pPFPMA length polymers (N=90, 190, 250) were further reacted with allylamine to amidate the polymer backbone and result in polymers decorated with allyl-alkenes. The advantage of this system is that it allows for modulation of the polymer system through highly efficient thiol-ene click chemistry. To achieve the polymer library, three polymerizations were first done with varying equivalence on monomer (20, 50, and 100 equivalents) while keeping the chain transfer agent (CTA):initiator ratios used set (1:0.05) to produce three polymer lengths. The monomer of PFPMA was mixed with 4-cyano-4-[(dodecylsulfanylthiocarbonyl) sulfanyl]pentanoic acid, and V-501 in 1,4-dioxane (4 M). The solution was degassed for 40 min via nitrogen purging before heating to 70° C. for 6.5 h under nitrogen positive pressure. The polymerization was quenched by cooling the reaction in liquid nitrogen and exposure to atmosphere. An aliquot was precipitated into pentanes and then filtered for pPFPMA NMR (1H and 19F) and attenuated total reflection-Fourier-transform infrared (ATR-FTIR) analysis. ¹H NMR (400 MHz, Chloroform-d) δ 2.25 (d, 2H), 1.52-0.99 (m, 3H). 19F NMR (400 MHz, Chloroform-d) δ −150.80 (2F), −156.99 (1F), −161.86 (2F). ATR-FTIR: 1778 cm⁻¹ (C═O, ester), 1518 cm⁻¹ (C=C, aromatic), 998 cm⁻¹ (C—O), 989 cm⁻¹ (C—O). The remaining pPFPMA was immediately reacted with a solution of allylamine and triethylamine mixture (2 equivalents to PFPMA monomer) through dropwise addition over 10 min. The solution was stirred at 50° C. under inert atmosphere for 16 hours. The reaction was quenched by cooling the reaction in ice. The obtained polymer was diluted and purified in a 1 kDa dialysis bag in methanol (MeOH) and further concentrated in vacuo to obtain a clear flaky yellowish-brown solid. ¹H NMR (400 MHz, dimethyl sulfoxide (DMSO)-d₆) δ 7.69 (s, 1H), 5.76 (s, 1H), 5.04 (s, 2H), 3.88 (d, 2H), 1.87 (s, 1H), 1.26 (s, 5H). ATR-FTIR: 1662 cm⁻¹ (C=C, allyl), 1632 cm⁻¹ (C═O, amide). Polymers were analyzed DMF SEC-MALLS (0.05 M LiBr).

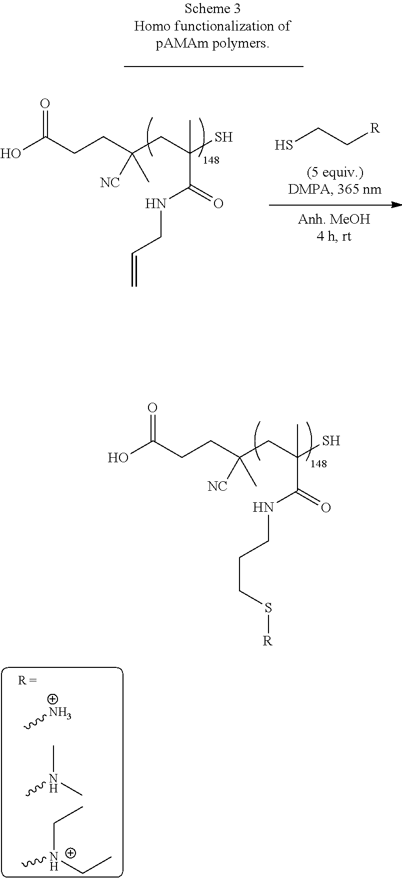

The different lengths of pAMAm (ru: 85, 150, 280, 1 equiv., 4 mmol relative to monomeric form, 0.5 g) were dissolved in MeOH (1 M, 4 mL) and mixed with one of three thiol-amine salts, cysteamine hydrochloride, captamine hydrochloride, diethylamino ethanethiol hydrochloride (5 equiv., 2.0 mmol), with 2,2-dimethoxy-2-phenylacetophenone (DMPA) photoinitiator (0.05 equiv) and run in an open capped 20 mL scintillation vial with a stir bar at room temperature in a UV (˜365 nm) gel nail curing box for 4 h. The resulting polymer solution was acidified with 0.5 mL HCl (1 M) and then purified in a 1 kDa dialysis bag in Millipore water and further concentrated via lyophilization to obtain a fluffy off-white powder. Cysteamine HCl functionalized polymer would collapse down overtime to a glassy looking tacky solid. Polymers were obtained at about 100-150 mg each polymer.

The scaffold pAMAm polymer (1 equiv., 0.8 mmol, 0.1 g) was mixed with cysteamine HCl (1 equiv., 0.8 mmol, 0.091 g), and DMPA photoinitiator (5 mol % relative to thiol) in DMSO-d₆ (0.1 M, 8 mL). One stock solution was made and then split into 10 samples and then reacted under UV light. Samples were taken out at varying timepoints (time=0, 0.25, 0.5, 1.0, 1.5, 2.0, 2.5, 3.0, and 4.0 h). Samples were then spiked with a stock solution of phenol in DMSO-d₆ to use as an internal standard. Timepoints were then measured by ¹H NMR to monitor alkene depletion over the 4 h window and was found that reaction kinetics were complete after 0.5 h.

The scaffold pAMAm polymer (1 equiv., 0.08 mmol, 0.01 g) was mixed with cysteamine HCl at varying equivalence (0.5, 1.0, 1.5, 2.0, and 3.0 equiv.), and DMPA photoinitiator (5 mol % relative to thiol) in -DMSO-d₆ (0.1 M, 0.8 mL) and then reacted under UV light for 1 h. Samples were then spiked with a stock solution of phenol in d-DMSO to use as an internal standard. Timepoints were then measured by ¹H NMR to monitor alkene depletion with an increase in thiol concentration by comparing to the phenol peak as well as the incorporation of the methylene peaks. To achieve full conversion, 5 equivalents of thiol were used in future experiments.

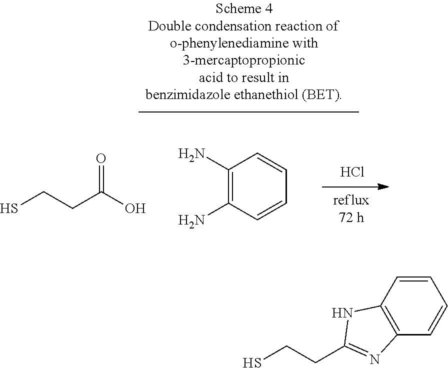 [SMILES: O=C(O)CCS ; Nc1ccccc1N ; Cl ; SCCc1nc2ccccc2[nH]1]

BET was synthesized by first adding 3-mercaptopropionic acid (3.214 mL, 36.9 mmol) to o-phenylenediamine (3.285 g, 30.4 mmol) following by refluxing in HCl (4 M, 15 mL) for 72 h. Solubility increased as the reaction continued. The mixture started out a deep orange/brown color and transitioned to a dark green. Crude reaction was taken off heat, and then diluted in a beaker with 200 mL nanopore H₂O. A 50/50 wt % sodium hydroxide (NaOH) solution was added dropwise to the beaker and white precipitates were formed. Precipitates were filtered and freeze dried to result in an off-white powder in BET (2.3 g, 42%). ¹H NMR (400 MHz, MeOH-d₄) δ 7.48 (nfom, 2H), δ 7.19 (nfom, 2H), δ 3.16 (t, 2H), δ 2.94 (t, 2H).

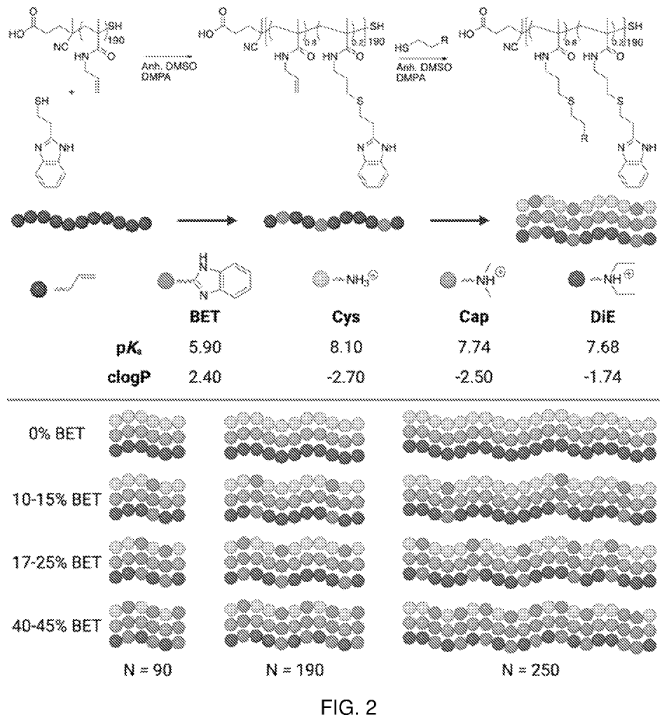

The polymer platform was first reacted with BET ranging in incorporation of 0-45%. After incorporation of BET and prior to purification, each resulting polymer was split into three vials and the remaining allyl alkenes were saturated with either cysteamine (Cys), captamine (Cap), or diethylamino ethanethiol (DiE) to result in a polymer designed with statistical incorporation of two amines with ranging pKa and logP (FIG. 2). To do so, the different lengths of pAMAm (ru: 90, 190, 247, 1 equiv., 1.2 mmol relative to monomeric form, 0.15 g) were dissolved in anhydride. DMSO (0.1 M, 12 mL) and each were mixed in three varying ratios (0.15 equiv., 0.18 mmol), (0.25 equiv., 0.3 mmol), and (0.5 equiv., 0.6 mmol) of BET, and then stirred with DMPA (0.05 equiv.) to be further reacted in an open capped scintillation vial with a stir bar at room temperature in a UV (˜365 nm) gel nail curing box for 1 h. The resulting partially BET functionalized polymers were then each split into 3 vials (4 mL each) and reacted further with one of three thiol-amine salts, cysteamine hydrochloride, captamine hydrochloride, diamino ethanethiol hydrochloride (5 equiv., 2.0 mmol) and reacted in the UV nail curing box in the same conditions with DMPA to result in 9 polymers starting from one pAMAm scaffold. The resulting polymer solution was acidified with 0.5 mL HCl (1 M) and then purified in a 1 kDa dialysis bag in Millipore water and further concentrated via lyophilization to obtain a fluffy off-white powder. Polymers with higher incorporation of BET are fluffier. Polymers were obtained between 70-90 mg per polymer. Polymer molar mass and dispersity measurements made on H₂O SEC-MALLS (0.1 M Na₂SO₄, 1% Acetic Acid).

The strength of this polymer platform is shown with the ability to synthesize a library of 36 polymers originating from one monomer to create three polymer backbone lengths, which further underwent step-wise post-polymerization modification to achieve a strategically designed library to systematically alter and analyze how polymer length, incorporation of BET, pKa, and hydrophobicity play a role into binding and delivering both plasmid DNA (pDNA) and CRISPR-Cas9 ribonucleoprotein (RNP).

The use of post polymerization modification has a few advantages in synthesis for this systematic analysis of variables. Rather than 36 separate polymerizations, this system allows for post modification of three polymer backbones to assure the polymer repeat units are the same when comparing the final polymer systems.

The platform also allows for the incorporation of functional groups that have previously proved difficult to polymerize, like imidazoles. BET is advantageous in gene delivery applications. The benzyl unit adds both hydrophobicity, which may be useful for complex aggregation and settling velocities for in vitro transfection, while adding the ability for possible intercalation for binding strength into pDNA. Imidazole's additionally have a lower pKa (˜5.0-6.0) which makes them advantageous for buffering within a more acidic biological region like the endo-lysosome. The incorporation of two amine types allows for binding at physiological conditions while the BET can amend as a further buffering amine at lower pH's to aid in endosomal escape. While the polymer library of Example 1 focuses on the use of 4 thiol-amines in functionalization, this technology of the pAMAm polymer scaffold is amendable for conjugation of a wide range of thiol based small molecules.

### Example 2: Characterization of the Polymers and Related Complexes

Example 1 demonstrates characterization of the polymer systems synthesized in Example 1 through ¹H NMR, ¹⁹F NMR, ATR-FTIR, and SEC-MALLS.

In polymerization of PFMPMA and further post-polymerization modification to pAMAm, ¹⁹F NMR is a helpful tool to probe the full conversion through amidation of allylamine by removal of the fluorine peaks. When coupling the thiols to the pAMAm scaffold, the decrease in allyl alkene peaks show full conversion, while the relative integrations of incorporation are measured by comparing the ratio of benzyl peaks in the BET to the methylene units incorporated of the cations. Three pAMAm scaffolds were synthesized for homopolymers of the Cys, Cap, and DiE cations (N=85, 150, 280), and three pAMAm scaffolds were synthesized for the remaining polymers with BET (N=90, 190, 250). The homopolymers were saturated using thiol-ene click chemistry using 5 equivalents of thiol amine. The polymers were synthesized in a stepwise conjugation of first BET while targeting 15, 25, and 45%, prior to splitting each vial into three and further saturating the remaining alkenes with Cys, Cap, or DiE. This process allows for the BET incorporation to be consistent across incorporated the various remaining cations for structural uniformity.

Offline batch mode measurements of ∂n/∂c of the polymers was made. A stock solution of polymer was made in H₂O (0.1 M Na₂SO₄, 1% Acetic Acid). Five dilutions were made in H₂O (0.1 M Na₂SO₄, 1% Acetic Acid) to achieve an order of magnitude difference with a concentration range of 0.2-2.0 mg/mL for Sh_Cys_0, Sh_Cap_0, Sh_DiE_0, and Sh_Cys_40. Samples were injected at a flow rate of 0.1 mL/min into a Wyatt Optilab T-rEX refractive index detector (25° C., λ0=660 nm). Refractive indices were measured at each concentration and the an/8c is determined from the slope. The four an/8c measurements were used to calculate an/ac's for the remaining polymers based on an equation from Striegel et al. Chromatographia (2017) 80:989-996. The following formula was used:

$${( \frac{\partial n}{\partial c} )}_{AB} = {{w}_{A} ( \frac{\partial n}{\partial c} )}_{A} + {{w}_{B} ( \frac{\partial n}{\partial c} )}_{B}$$

Altering the protonation state and hydrophobicity of polymer systems has been shown to affect the polymer's ability to bind, release, and settle on cells in an in vitro setting. This polymer set allows for systematic changes in a range of pKₐ values of the cation and ranging logP values.

The pKₐ values were measured via an autotitrator of the small molecule thiols. To do so, each small molecule thiol was titrated to identify the pKₐ of the amine. The small molecule thiol of BET, cysteamine hydrochloride, 2-(dimethylamino) ethanethiol hydrochloride, and 2-(diethylamino) ethanethiol hydrochloride were diluted to a 1 mg/mL solution in 30 mM HCl. The samples were further titrated with 75 mM NaOH in an auto-titrator, Orion star T901 pH titrator. BET: 5.90, DiE: 7.68, Cap: 7.74, Cys: 8.10. The pKₐ value was calculated using the following formula:

$$Polymer {pK}_{a} = \frac{( RU ) ( {pK}_{a} BET ) ( clogP BET ) + ( RU ) ( mol cation ) ( {pK}_{a} cation )}{RU}$$

The pKa analysis gave values shown in FIG. 2 of 8.10, 7.74, 7.68, and 5.90 for Cys, Cap, DiE, and BET respectively. The polymers were unable to show two distinct pKa's, but rather one long buffering capacity. Due to counter ion effect, it is common to see one pKa unit drop from monomer to polymer form for amine-based cations. Additionally, a homopolymer of BET was insoluble in water and therefore was unable to be titrated.

To understand the hydrophobicity, the calculated octanol to water coefficient (clogP) values of the same small molecules were analyzed computationally. To do so, calculations for clogP values were completed by Molinspiration Property Calculation Service of the Molinspiration Cheminformatics website. The following formula was used to calculate clogP:

$$Polymer clogp = \frac{( RU ) ( mol BET ) ( clogP BET ) + ( RU ) ( mol cation ) ( clogP cation )}{RU}$$

The analysis gave clogP values of −2.70, −2.50, 1.74, and 2.40 for Cys, Cap, DiE, and BET respectively; where a more negative number is more water soluble and more positive is more likely to be in octanol.

Molar averages of pKa and clogP of the polymer systems were calculated based on the repeat units and percent incorporation of the functional moieties. By calculating the average polymer pKa and clogP, the polymer library is able to be analyzed to see how charge and hydrophobicity changes can affect future expression outputs.

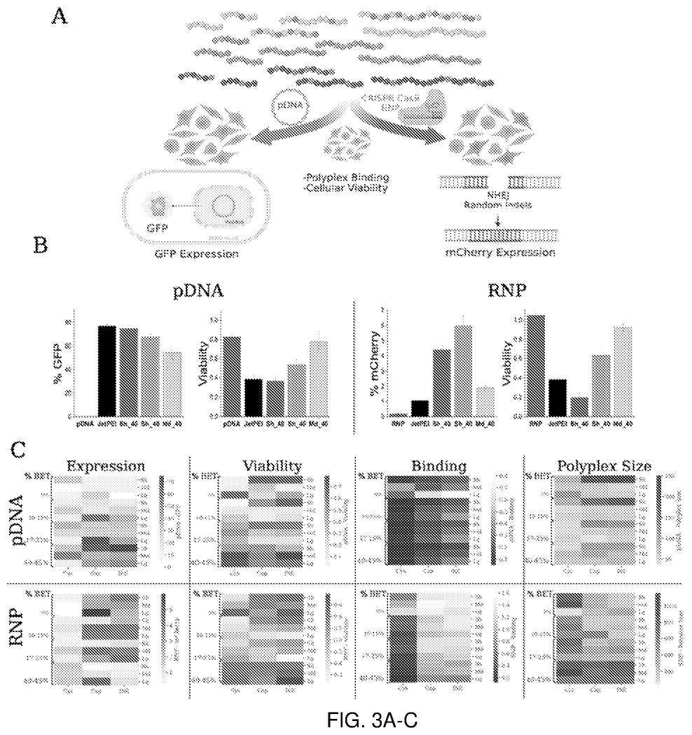

Characterization of the polymer-payload complexes were analyzed by dynamic light scattering (DLS) and a dye exclusion assay to understand the size and the compaction degree of the complexes (FIG. 3C). To understand the hydrodynamic radius (Rh) of the complexes, the polymers were mixed with either pDNA or RNP in phosphate buffered saline (PBS, pH 7.4). Polymer and pDNA solutions were mixed at a nitrogen on polymer to phosphorous on pDNA ratio (N/P ratio) of 10 and 20 to mimic initial biological screening. The polymers with the medium and large backbone length hovered just above 50 nm in size, while the small backbone length polymers generally had a larger Rh ranging from 50-250 nm in size. When the polymers were bound with RNP at an N/P ratio of 5, the complex sizes were considerably larger with none of the complexes being smaller than 100 nm and ranging to particle sizes around 1000 nm. The RNP aggregates alone and could be cause for these larger aggregates in PBS solution. Complex size is often related to aggregation and settling speeds of these complexes, which can in turn effect internalization mechanisms.

Nucleic acid compaction can be measured through a dye exclusion assay in which an intercalating dye is excluded and loses fluorescence after a polymer competitor is bound. PicoGreen was mixed with pDNA prior to polymer addition to form complexes at N/P 10 and 20. A trend of lower fluorescence and therefore tighter compaction can be observed with less bulkiness in the increasing degree of compaction order of DiE, Cap, and Cys. Additionally, it can be seen that the more incorporated BET, the lower the fluorescence as well, indicating possible intercalation within the pDNA. What also can be observed is that the shorter the polymer backbone, the tighter binding, which can be attributed the difficulty of the larger polymers to be flexible and conform to each groove of the rigid pDNA, while the short backbone length is able to exclude more PicoGreen due to chain flexibility. When these same polymer systems at N/P 5 were bound with the RNP complexes using OliGreen as the excluding dye, similar trends in regard to the Cys and BET functionalization were seen. Cys functionalized polymers showed the tightest binding and the increased BET incorporation displayed tighter binding trends to the shorter gRNA in the RNP complex. However, in this case a slight trend towards larger polymer backbone size increased the amount of dye exclusion, showing an opposite trend of the pDNA of polymer length and tightness of binding. While efficient binding is important in most cases, strong binding may not be linked directly to efficient transfection, as it is also important to release these biological payloads.

Characterization of the polymers and complexes aids in understanding how the physical characteristics affect the final transfection efficiency. Thus, the polymer library synthesized in Example 1, and characterized in Example 2, compare the chemical and physical characteristics of polymer backbone repeat units, type of cation, incorporation of BET, polymer pKₐ, polymer clogP, complex size, and complex compaction, in order to identify structure property relationships to transfection efficiency.

### Example 3: Characterizing Complex Delivery to Cells

Example 3 demonstrates the characterization of the present polymers in delivering biological agents to cells.

Measuring a fluorescence output using the delivery of biological payloads are effective tools to explore new polymer platforms for delivery. A common delivery payload for initial screening is the delivery of pDNA that exhibits transient expression of an enhanced green fluorescent protein (GFP), which can be measured on a per cell count through flow cytometry. In situ cell health and cell metabolism were also monitored through a CCK8 viability assay with the same transfection as used to measure expression 48 h post transfection. An initial screen of the polymer library was completed by delivering the GFP encoded pDNA to HEK293T cells at an N/P of 10 and 20 (FIG. 3A).

The initial pDNA screen was completed over three separate transfections with twelve polymers each in triplicate, and the percent of cells were normalized across all the transfections based on the average JetPEI percent cells expression. An initial screen of the library was also completed delivering RNP, composed of a complex formed with gRNA and CRISPR-Cas9 protein.

The HEK293T cell line was cultured in high glucose DMEM with added 10% HI—FBS and 1% Antibiotic/Antimicrobic. The incubator was set to 37° C. with 5% CO₂ and under humidified atmosphere. Cell confluency was monitored, and cells were passaged as needed. Cells were plated in a 48-well plate format at a density of 50,000 cells/mL.

HEK293T cells were plated in polystyrene 48-well plates at a density of 50,000 cells/mL in DMEM (10% HI—FBS). After 24 h, complexes with pDNA were prepared in H₂O by adding 80 μL polymer to 80 μL pDNA (0.02 μg/mL) at molar ratios of polymer to get N/P ratios. Complexes with RNP were prepared in PBS by adding 80 μL polymer to 80 μL RNP (gRNA (0.02 μg/mL) and spCas9 protein (0.1 μg/mL) complex) at molar ratios of polymer to get N/P ratios. Complexes were allowed to form at room temperature for 40 min prior to the addition of Opti-MEM in a 2:1 ratio (320 μL) to the complexes immediately before addition to cells. Media was carefully aspirated from the well plate before addition of the complex samples. Each complex was split into triplicate adding 150 μL to each well. Well plates remained on the bench top at room temperature for 40 min before placing into the 37° C. incubator (5% CO₂). 4 h after initial transfection, 1 mL of DMEM (10% HI—FBS) was added to each well. Media was further aspirated 24 hours after initial transfection, and fresh DMEM (10% HI—FBS, 1 mL) was added to each well. The cells were analyzed for CCK-8 and flow cytometry analysis 48 hours following initial transfection.

Quantification of transfection efficiency with the full polymer library of HEK293T cells delivering pDNA encoding for GFP were measured via flow cytometry. Following 48 h after initial transfection, cells were harvested for flow cytometry to quantify percentage of GFP or mCherry positive cells. To harvest cells, media was aspirated, HEK293 cells were trypsinized (150 μL), and added to a V-shaped 96-deepwell plate to be centrifuged at 4° C. The supernatant was aspirated away, and cell pellet was resuspended with calcein violet live-cell stain in PBS with 1% FBS. Cells incubated with the cell stain for 30 min on ice without light before measuring via flow cytometry for GFP or mCherry. 405 nm (calcein violet) and 488 nm (GFP) lasers were used on the flow cytometer (ZE5, Biorad, Inc., CA, USA). At least 5,000 events were collected for every treatment in triplicate.

Cell counting kit-8 (CCK-8) was used to measure cell viability after transfection. Transfection procedures were carried out as written in in vitro cell transfection using complex section. 48 hours post initial transfection, media was aspirated. FluoroBrite (0.5 mL) containing 10% FBS was mixed with CCK-8 dye (40 μL) and added to each well to incubate for 2 h. After incubation, 150 μL of the supernatant was removed and placed in a 96-well plate and absorbance was measured at 450 nm using a Synergy H1 Hybrid Reader. The untreated cells were normalized to 1.0 cell survival.

The small molecule PicoGreen dye was incubated for 15 min with pDNA (0.02 mg/mL) and was diluted to a final concentration with a ratio of 1:200 in H₂O. Complexes were prepared in H₂O by adding 170 μL polymer to 170 μL Pico Green-pDNA (0.02 mg/mL) at various molar ratios of polymer to get N/P ratios of 10:1 and 20:1. Complexes were allowed to form at room temperature for 40 min and then split into triplicate by separating the complex solution into three wells of 100 μL each in a 96 well plate. Fluorescence of PicoGreen was measured (excitation: 485 nm, emission: 528 nm) using a Synergy H1 Hybrid Reader. Since PicoGreen only fluoresces when intercalated with the pDNA, the lack of fluorescence signifies exclusion and thus, polymer binding to the pDNA. Samples were normalized in comparison to pDNA and PicoGreen with no competitive polymer. A blank control of PicoGreen in water was also run and subtracted from each sample to account for autofluorescence/background. Dynamic light scattering (DLS) data of the three backbone functionalized polymers that are mixed with pDNA to form complexes. The complexes were measured by DLS in order to better understand the hydrodynamic radius (Rh). All PBS (pH=7.4) was pre-filtered through a 0.2 μm GHP syringe filter and prepared to run in a high-throughput DLS DynaPro Plate Reader III. For high-throughput DLS, samples were transferred into a glass-bottomed 96-well DLS plate. The well plate was placed in the DynaPro Plate Reader III and equilibrated at 25° C. Complexes were prepared in PBS by adding 100 μL polymer to 100 μL pDNA (0.02 μg/mL) at a molar ratio of polymer to achieve N/P ratios of 10:1 and 20:1. Wells containing samples of interest were analyzed using automated measurements after letting the complex form for 40 min in the well. For each measurement, five acquisitions were recorded with an acquisition time of 5 seconds each. The hydrodynamic radii of complexes from RNP and backbone functionalized polymers in PBS (pH=7.4) were measured by DLS with a Zetasizer Nano Series (Malvern Panalytical, Westborough, MA). PBS was pre-filtered through a 0.2 μm syringe filter. To prepare RNP, 30 μL of gRNA (0.04 mg/mL) was added to 30 μL of spCas9 (0.2 mg/mL) and allowed to form complexes for 20 minutes. To form complexes, 60 μL of polymer solution at a molar ratio of polymer to achieve an N/P ratio of 5 was added. The complexes were allowed to form for 45 minutes and diluted with 240 μL of PBS. Samples were placed in disposable microcuvettes, and 3 measurements taken using 1730 backscatter.

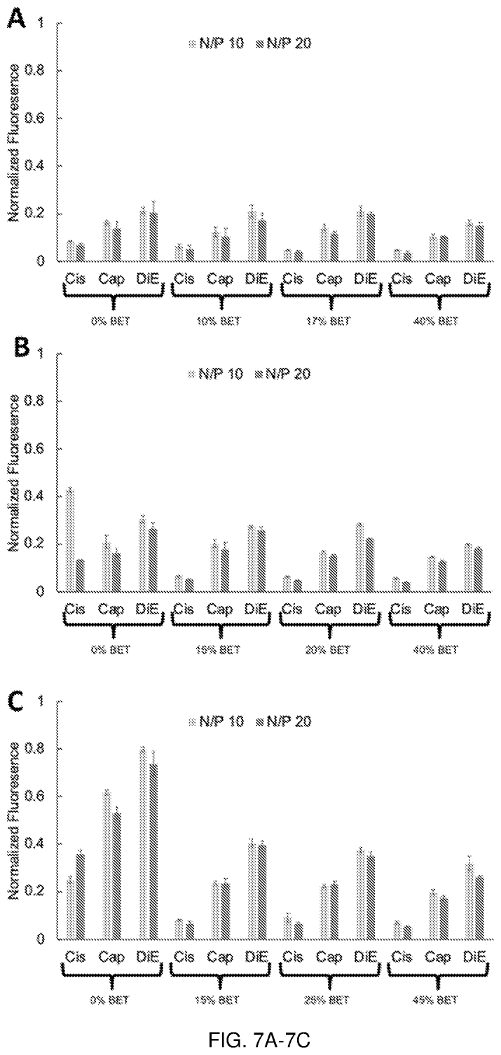

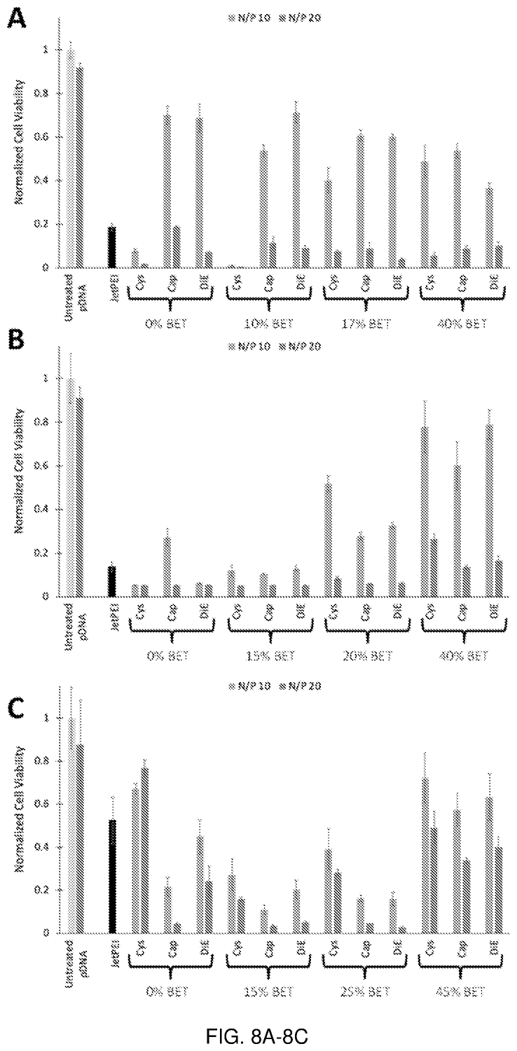

Regarding the pDNA, what can be observed is that for the short and medium backbone length polymers, an increase in BET incorporation increased the transfection efficiency with the most effective polymer being Sh_DiE_40 (polymer length_cation_BET incorporation) with 75% GFP positive cells (FIG. 3B, FIG. 7A,B). The commercial control of JetPEI exhibited 77% GFP positive cells. The large backbone polymer had the most polymers above 40% GFP positive cells (FIG. 7C). At an N/P of 20, most polymers were unhealthy and existed with a low cell viability (FIG. 8A-C). At an N/P of 10, the short backbone length exhibited the highest viability (FIG. 8A). This initial screening was able to identify polymer systems within the library that were more effective at delivering pDNA and based on the comparative balance of expression and toxicity, further analysis was suited at more optimized N/P ratios.

Regarding the delivery of RNP, instead of transient protein expression, as seen from the pDNA reporter assay, the RNP complex is set to undergo a precise gene editing function on the cell's chromosome. An HEK293T cell line was engineered with a traffic light reporter (TLR) gene, which allows for the quantification of nonhomologus enjoining (NHEJ) editing through a DNA-repair mechanism which results in the expression of mCherry. Through the imprecise repair pathway, it is known that only about ⅓ of the NHEJ repair mutations produce the proper frameshift to produce mCherry. While imperfect, the TLR cell line offers an efficient and high-throughput method to analyze NHEJ editing through flow cytometry, rather than the laborious and time-consuming DNA sequencing. The full polymer library was complexed with RNP and initially screened at an N/P of 2.5 and 5; the mCherry expression and CCK8 viability was measured at 48 h, alike the pDNA transfection (FIG. 3A). Despite that the best compaction of RNP was seen with the Cys cationic functional group, Cap and DiE showed significantly more mCherry expression than the primary amine alternative with the highest mCherry expression being Lg_Cap_0 at 6% with N/P 5 (FIG. 3B). The commercial control of JetPEI exhibited 1.04% mCherry positive cells, a six-fold decrease from the most efficient polymer of interest. It was able to be seen that an increase in BET incorporation did not correlate to increased expression, as was seen more distinctly in the pDNA delivery. Additionally, the increase in backbone size trended towards higher NHEJ editing. Viability of the cells when transfected with the polymer library all showed significantly higher metabolisms than the JetPEI control at N/P 2.5 and 5, thus polymers exhibited lower levels of toxicity. Given the improvement in RNP delivery in comparison to the JetPEI control, the present polymer systems were able to be identified as promising vectors.

Initial screening of the polymers allows for the wide screening of large polymer libraries to understand polymers of interest for a specific biological payload, pDNA or RNP (FIG. 3C).

### Example 4: Sequential Optimization of Expression Outputs Guided by Machine Learning

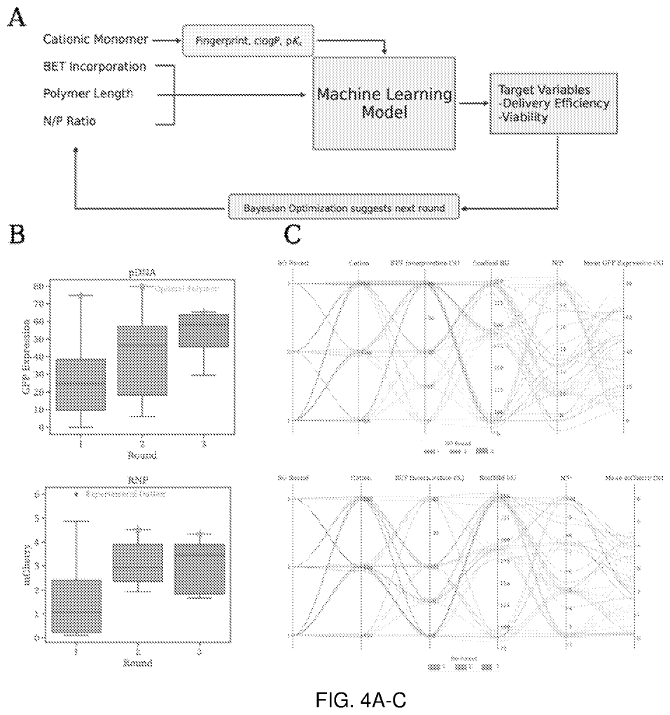

Previously, it could be a hurdle to effectively identify and optimize polymer sets that lead to good delivery efficacy from large combinatorial polymer libraries. In the case of polymer library synthesized in Example 1, cation incorporation, BET incorporation (%), polymer length (scaffold RU) and N/P ratios were variables. Despite a limited number of discrete experimental choices for these design variables, there may be hundreds or thousands of polymer formulations to explore for a given delivery cargo, e.g., pDNA or RNP. A uniform or quasi-random sampling (e.g., using design of experiments) of the variable space may hide potentially high performing polymers or may require a large number of samples to identify the best performing polymers. To efficiently explore the combinatorial design space and its impact on expression outputs, batch Bayesian optimization (BO) was used. According to this machine learning technique, a probabilistic model was learned, which is herein called the “the optimization model” (FIG. 4A), that relates the design variables to the target delivery efficacy. This optimization model is advantageous in this sequential optimization task in discrete or mixed polymer design spaces. Then, based on the predicted mean and variability of the target variable in the design space, a new batch was chosen, which is herein called the BO round, of promising polymers to formulate and measure transfection efficacy. The new batch balanced exploitation, sampling similar polymers to the best performing polymers thus far, and exploration, exploring areas of the design space with more uncertainty that may lead to new good performing polymers. This process was repeated several times leading to a subset of optimal polymers in the variable space (FIG. 4A). In this work, based on the dataset for both the pDNA and RNP delivery produced in Example 3, two additional rounds of transfections were completed with the most promising polymer subsets at specific N/P ratios. In order to complete this, based on the Example 3 data set for both the pDNA and RNP delivery (called Round 1), two additional rounds of transfections were completed with select polymer subsets at specific N/P ratios.

Guided by machine learning, Round 2 involved the use of twelve total polymers at two N/P ratios, six delivering pDNA (N/P 7.5, 12.5) and six for RNP (N/P 7.5, 10). Complex formation, transfection, CCK-8 analysis, and flow cytometry workup were all followed as listed above in the general procedures. For pDNA delivery, polymers of Sh_DiE_40, Sh_Cap_40, Lg_Cap_25, Md_Cys_40, Md_DiE_40, and Lg_DiE_45 were tested at N/P ratio of 7.5 and 12.5. For RNP delivery, polymers of Lg_Cap_0, Lg_Cap_45, Md_Cap_15, Md_Cap_20, Md_DiE_15, and Sh_DiE_0 were tested at N/P ratio of 7.5 and 10.

Round 3 then included a transfection of eight polymers at distinct N/P ratios for each polymer and each biological cargo. Complex formation, transfection, CCK-8 analysis, and flow cytometry workup were all followed as listed above in the general procedures. For pDNA delivery: Sh_DiE_40 (N/P 11.25, 14.125, 16.375), Lg_Cap_20 (N/P 11), Lg_DiE_40 (N/P 17.5), Lg_Cap_40 (N/P 13.25), Lg_DiE_20 (N/P 13), Md_DiE_40 (N/P 17.125). For RNP delivery: Lg_Cap_0 (N/P 4, 5.5, 6), Sh_DiE_20 (N/P 8, 10), Lg_Cap_20 (N/P 9.625), Lg_Cap_10 (N/P 8.625), and Lg_DiE_20 (N/P 8.875). After three optimization rounds, no further improvement was observed in the expression targets and the sequential optimization was deemed concluded.

The progression of FIG. 4B presents expression outputs for pDNA and RNP cargos respectively. Each box plot corresponds to the measured outputs after transfection. For pDNA in FIG. 4B, after broad initial exploration in Round 1, the model suggested a subset of better performing polymers in this new round, reaching the overall best polymer for the dataset. In Round 3, the higher mean GFP expression indicates the model exploits a subset of polymers with higher expression but fails to produce an overall optimal polymer across the whole dataset. In the case of RNP, the model explored a subset of promising polymers in Rounds 2 and 3, which outperformed most of the initial polymer choices, but failed to outperform two data points that were collected at Round 1. The NHEJ editing resulted in mCherry positive cells from the RNP is relatively low. The parallel coordinate plots (FIG. 4C) summarize the progression of the sequential optimization to identify subsets of polymers that result in the highest expression. For pDNA (FIG. 4C, top), the model directed experimental sampling towards the cations of Cap and DiE, higher BET incorporations, and intermediate N/P ratios than the initial screen of 10 and 20, with top three preforming polymers being Sh_DiE_40, Sh_Cap_40, and Lg_Cap_25. For RNP (FIG. 4C, bottom), the model suggested sampling cations with Cap and DiE, lower BET incorporations, and polymers derived from the larger scaffolds, with top three preforming polymers being Lg_Cap_0, Lg_Cap_15, and Sh_DiE_0. These top resulting design choices provided the experimentalist with information to make future modifications of the design and synthesis space dependent on the biological payload of choice.

### Example 5: Explanation of Expression Relationships Guided by Machine Learning

Example 5 demonstrates optimization of expression outputs of the present complexes using machine learning.

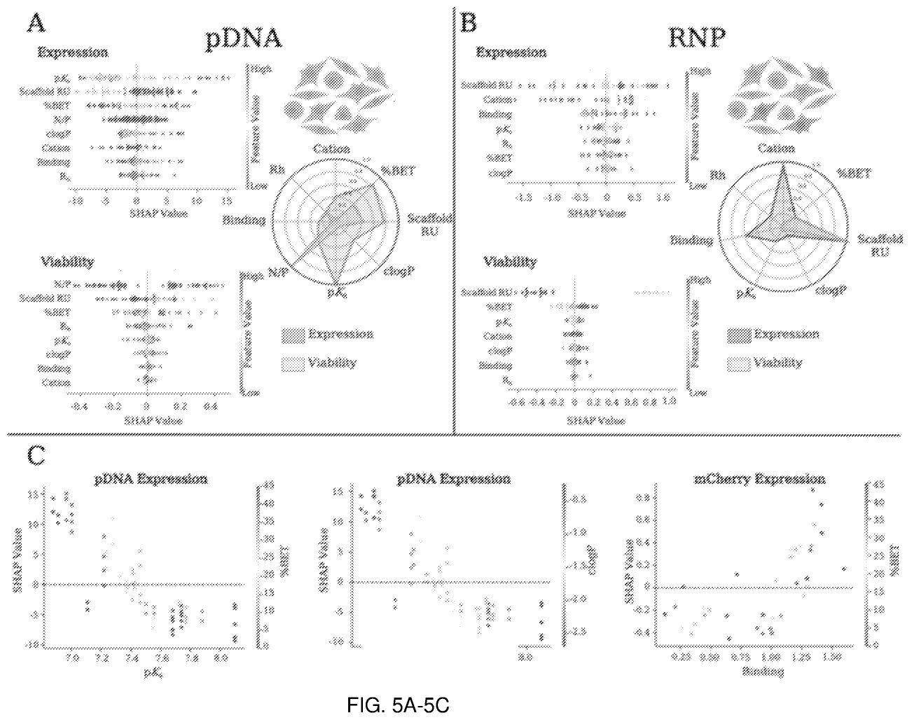

Elucidating the correlation between physico-chemical parameters of polymer vehicles and the delivery efficacy or cell viability is challenging. To explore structure-property relationships, the approach in Kumar et al. was followed, in which a second machine learning model was fit, herein called an “explanation model”. The characterized variables of polymer backbone repeat units, type of cation, incorporation of BET, polymer pKₐ, polymer clogP, complex size (Rh), and complex compaction were used to model their effect on efficacy and viability. SHAP values were computed to extract the predictive importance of each polymer or complex characteristics on these output variables. These values and model explanations were analyzed for the delivery of both pDNA (FIG. 5A) and RNP (FIG. 5B) biological payloads.

For each datapoint (polymer) in the dataset, a SHAP value was generated, wherein SHAP is a measure of how much the magnitude and change of a given variable affects the output of the explanation model. Thus, a high absolute SHAP value is correlated to high impact on the output variable according to our explanation model. A positive SHAP value is translated into a positive effect, and a negative SHAP value has a negative effect on the output variable. This is not a causal effect but an explanation of how the variables affect the plausible model of transfection efficacy and viability. For pDNA, polymer pKa, BET incorporation, and scaffold repeat unit had the highest absolute SHAP values aggregated across samples, which are translated into impact on GFP expression. Lowering the pKa had a positive impact on GFP expression, hypothesized due to the better buffering capacity when inside the endosome, thus leading to endosomal escape. The decrease in the polymer pKa is a result of an increase in BET incorporation which has two amines, one cationic which is readily available for binding at physiological pH, and the other being BET which can protonate at lower pHs. The SHAP dependency plots (i.e. SHAP values as function of a given feature) of pDNA expression (FIG. 5C: left, middle) show a linear dependence correlating a lower pKa, increasing BET incorporation, and increasing clogP value trend to higher SHAP values. Regarding scaffold repeat unit, surprisingly, the largest impact on GFP expression was through either the small backbone or large backbone size, while the medium length was the least effective. As mentioned previously incorporation of BET implements aspects of increased hydrophobicity, a lower pKa functional amine, and an additional binding mechanism to electrostatics, in pDNA intercalation. It was seen that higher percentages of BET incorporation showed to have a positive correlation with GFP expression and interestingly also cell viability. Often, higher incorporations of hydrophobic units can increase internalization through membrane disruption and thus cause higher toxicity, which is not seen here. This signifies that the functional aspect linked with the incorporation of BET, is most linked to the lower pKa of the imidazole (5.90). Average SHAP values correlate to the level of impact each polymer feature has on the output of GFP expression or cell viability when delivering pDNA.

When analyzing the delivery of RNP and looking at the expression output of mCherry (FIG. 5B), only one polymer component of the top three most important features was shown to impact pDNA, scaffold repeat units. For RNP delivery there was a linear correlation seen with increasing backbone scaffold repeat units and an increase in mCherry expression. Polymer cation (where Cys=0, Cap=1, DiE=2) showed how Cys showed the least impactful in GFP and mCherry expression while RNP delivery favored the Cap cation in the polymer for delivery. An additionally surprising aspect was that complexes with higher dye exclusion values (binding), which attributes to looser compaction, displayed higher correlations to mCherry expression, and thus the tighter the binding, the less impactful the expression output. This can be further seen in a dependency plot comparing how binding and BET incorporation impact mCherry expression (FIG. 5C, right). A lower compaction is correlated with lower BET incorporation and thus higher SHAP values. This BET incorporation trends oppositely from pDNA transfections, which can show the intricacies in tuning polymer delivery vectors based on the biological payload. Average SHAP values correlate to the level of impact each polymer feature has on the output of mCherry expression or cell viability when delivering RNP.

Analyzing the polymer systems through SHAP analysis allows for a more detailed understanding of which components are most influential for gene expression. Within this system it was seen that lower pKa in conjunction with higher BET incorporation were perhaps the most indicative of pDNA expression, while a larger scaffold size, incorporation of cation type of Cap, and surprisingly lower BET incorporations were perhaps the most influential for RNP expression. This analysis not only aids in analysis within this specific combinatorial library, but also allows for insight into fundamental polymer properties that may aid in future delivery vectors.

### Example 6: In Vivo Tail Vein Delivery of pDNA into Mice

Example 6 demonstrates in vivo delivery of pDNA into mice using the present polymers.

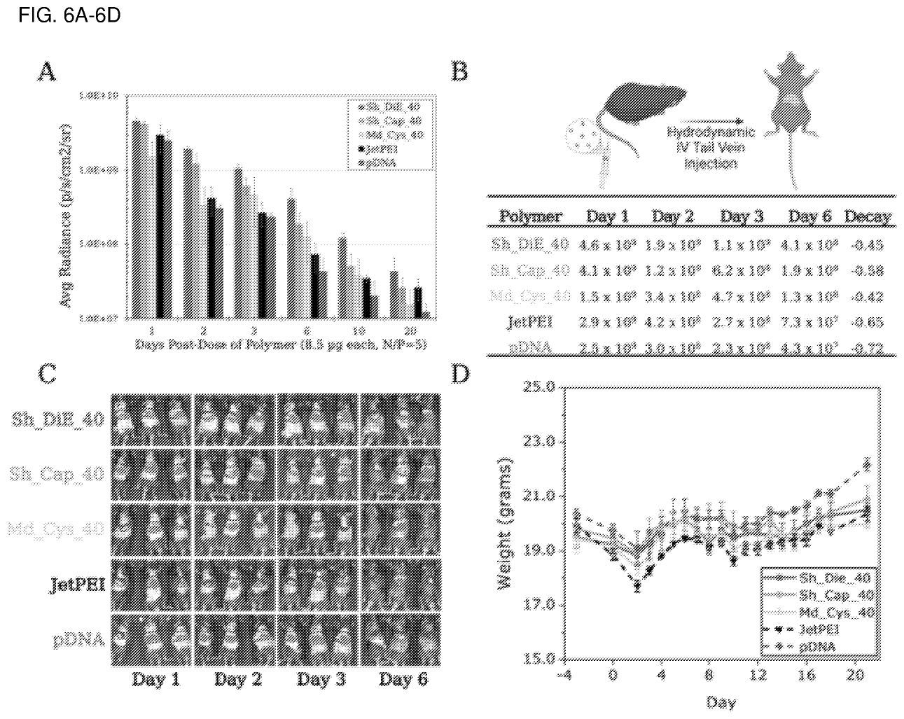

Top preforming polymers were further investigated via in vivo transfection into mice. While polymeric gene-delivery vectors have proved to be effective in vitro, the largest hurdle for non-viral gene delivery is in vivo efficiency. The top preforming pDNA in vitro expressing polymer of each cationic class was selected (Sh_DiE_40, Sh_Cap_40, Md_Cys_40) and formed with a luciferase expressing pDNA (8.5 μg) at N/P of 5 in D5W. The complexes were administered via hydrodynamic tail vein injections (1.8 mL) in triplicate mice and compared to the controls of naked pDNA and JetPEI complexes. Mice were injected with luciferin and the average radiance was measured over 20 days. Naked pDNA has shown precedence to have high expression of luciferase through hydrodynamic injections due to an injection of a relatively large volume (5-10 wt %) over 4-8 seconds, inducing a high pressure plug that localizes to the liver. While hydrodynamic injections can be stressful, all mice survived the tail vein injection, and it was seen that the weight of the mice stayed relatively healthy over the 20-day span (FIG. 6D). Mice injected with JetPEI complexes saw the largest dip in weight in the first 48 h but recovered after about 6 days. The three polymers of interest saw a smaller dip in weight over the first 48 h, increase in weight prior to injection at 96 h and hovered around the starting weight over the 20-day span. Kinetic expression of the hydrodynamic injection was understood through measuring expression on day 1, 2, 3, 6, 10, and 20 post injection (FIG. 6A, FIG. 6B, and FIG. 6C). While naked pDNA initially showed high expression as expected, it had the fastest decay rate of expression over the timespan, and 1.7 folder faster than Md_Cys_40 and 1.6-fold faster than Sh_DiE_40 (FIG. 6B). All three polymers showed a slower decay rate over a six-day period that both the controls of JetPEI and pDNA. These results show that the complexes were able to protect the pDNA from being secreted more rapidly. After day 2, the naked pDNA exhibited the lowest radiance (p/s/cm²/sr). Over the first 10 days, all three polymers of interest outperformed JetPEI in average radiance. Md_Cys_40 decayed at a faster rate between day 10 and 20, dipping below JetPEI. Polymers Sh_DiE_40 and Sh_Cap_40 outperformed JetPEI and showed significant increases in expression over the naked pDNA. Sh_DiE_40 showed the highest radiance throughout and ranged 0.5-0.8 order of magnitude larger over JetPEI and pDNA displaying as an efficient delivery vector. These results show that the present polymers can bind, protect, and deliver pDNA to the liver. These results also show that the present polymers have higher delivery efficacy than a JetPEI commercial control.

Other embodiments are in the claims.

What is claimed is:

1. A polymer of formula (I) 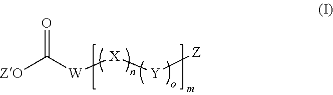
wherein
W is alkylene;
each X is independently 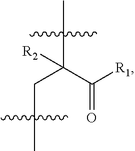
wherein R₁ is —O—C₆F₅ or —NR′R″, wherein R′ and R″ are each independently H, alkenyl, or substituted alkylthio alkyl, and wherein R₂ is H or CH₃;
each Y is independently 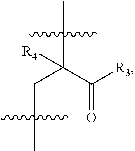
wherein R₃ is —NR′R″, wherein R′ and R″ are each independently H, alkenyl, or substituted alkylthio alkyl, and wherein R₄ is H or CH₃;
Z is hydrogen, alkyl, thiol, or 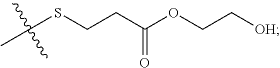 [SMILES: *SCCC(=O)OCCO]
is H, or alkyl;
m is from 1 to 1000;
each of n and o represents a fraction of m, and the sum of n and o is 1;
or an ion or salt thereof.

2. The polymer of claim 1, wherein at least one X or Y is 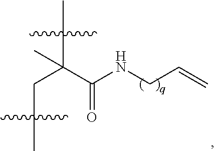
wherein q is 1 to 4.

3. The polymer of claim 1, wherein at least one X or Y is 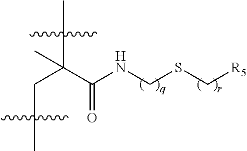
wherein R₅ is NRₐR_{b}, where Rₐ and R_{b} are independently H or alkyl, and q and r are independently 1 to 6.

4. The polymer of any of claims 1 to 3, wherein at least one X or Y is 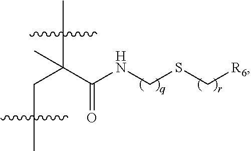
wherein R₆ is a basic nitrogen containing heterocycle, and q and r are independently 1 to 6.

5. The polymer of claim 4, wherein the basic nitrogen containing heterocycle is

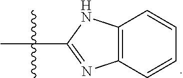 [SMILES: *c1nc2ccccc2[nH]1]

6. The polymer of any of claims 1 to 5, wherein W is substituted with cyano.

7. A compound of formula (II) 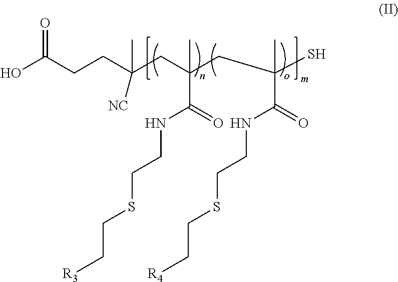
wherein
R₃ is NH₃, N(CH₃)₂, or N(C₂H₅)₂;
R₄ is a basic nitrogen containing heterocycle;
m is from 1 to 1000;
each of n and o represents a fraction of m, and the sum of n and o is 1;
or a salt or ion thereof.

8. The polymer of claim 7, wherein the basic nitrogen containing heterocycle is

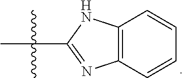 [SMILES: *c1nc2ccccc2[nH]1]

9. The polymer of any of claims 1 to 8, wherein m is from 1 to 500.

10. The polymer of claim 9, wherein m is from 50 to 300.

11. The polymer of any one of claims 1 to 10, wherein n is from 0.5 to 1.

12. The polymer of claim 11, wherein n is from 0.7 to 1.

13. A complex comprising a first polymer of any one of claims 1 to 12 and a negatively charged biological agent.

14. The complex of claim 13, further comprising a second polymer of any one of claims 1 to 12.

15. The complex of claim 13, wherein the negatively charged biological agent comprises a nucleic acid.

16. The complex of claim 13, wherein the nucleic acid comprises DNA or RNA.

17. The complex of claim 15, wherein the nucleic acid comprises gRNA, mRNA, tmRNA, tRNA, rRNA, siRNA, shRNA, PNA, ssRNA, dsRNA, pDNA, ssDNA, dsDNA, a DNA:RNA hybrid molecule, a plasmid, an artificial chromosome, cDNA, a PCR product, a restriction fragment, a ribozyme, an antisense construct, or a combination thereof.

18. The complex of claim 13, wherein the negatively charged biological agent comprises a protein.

19. The complex of claim 18, wherein the protein comprises a ribonucleoprotein.

20. The complex of claim 19, wherein the ribonucleoprotein comprises a virus, a ribosome, telomerase, Ribonuclease P (RNase P), a heterogeneous ribonucleoprotein particle (hnRNP), or a small nuclear ribonucleoprotein particle (snRNP).

21. The complex of claim 18, wherein the protein comprises a nuclease.

22. The complex of claim 21, wherein the nuclease comprises a zinc finger nuclease (ZFNs), a transcription-activator like effector nucleases (TALEN), or a Cas protein.

23. The complex of claim 22, wherein the Cas protein comprises Cas2, Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, CjCas9, SpCas9, Cas12, Cas13, Cas14, CfpI, CasI, CasIB, Cpf1, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, Cu1966, modified versions thereof, or combinations thereof.

24. The complex of claim 22, wherein the Cas protein is Cas9.

25. The complex of claim 13, wherein the negatively charged biological agent comprises a nucleic acid and a nuclease.

26. The complex of claim 25, wherein the negatively charged biological agent comprises gRNA and a Cas protein.

27. The complex of any one of claims 13 to 26, wherein the negatively charged biological agent is bound noncovalently to the polymer.

28. The complex of any one of claims 13 to 26, wherein the polymer is complexed with the negatively charged biological agent.

29. A composition comprising the complex of any one of claims 13 to 28 and a liquid carrier.

30. A method comprising contacting a cell with the complex of any one of claims 13 to 28, wherein the biological agent is delivered into the cell.

31. A method of identifying polymers for nucleic acid delivery comprising
(i) determining a SHAP value for at least one of scaffold RU, cation type, % or type of hydrophobic monomer, polymer pKₐ, polymer clogP, polyplex size (Rₕ), formulation (N/P) ratio, and binding strength for a plurality of polymers;
(ii) selecting monomers for a polymer based on the magnitude and sign of the SHAP value.

32. The method of claim 31, wherein the polymer is a polymer of claim 1.

33. The method of claim 31, wherein the % hydrophobic monomer is % BET.

34. The method of claim 31, further comprising synthesizing the polymer and assaying the polymer for expression of a cargo or viability.

35. A method of identifying polymers for nucleic acid delivery comprising
(i) providing a machine learning model trained with data from a plurality of polymers; and
(ii) using Bayesian optimization in the machine learning model to identify the polymers.

36. The method of claim 35, wherein the polymer is a polymer of formula (I).

37. The method of claim 36, further comprising synthesizing the polymers and assaying the polymers for expression of a cargo or viability.

38. The method of claim 37, wherein data from the assaying is used to retrain the machine learning model, and the method further comprises using Bayesian optimization in the retrained machine learning model to identify additional polymers.

39. The method of claim 35, wherein the data includes data on scaffold RU, cation type, % or type of hydrophobic monomer, polymer pKₐ, polymer clogP, polyplex size (Rₕ), formulation (N/P) ratio, binding strength, expression level, and/or viability for the plurality of polymers.
